(19) United States
(12) Patent Application Publication
(10) Pub. No.: US 2007/0293491 A1
(43) Pub. Date: Dec. 20, 2007

(54) INDAZOLE COMPOUNDS AND METHODS FOR INHIBITION OF CDC7

(75) Inventors: Cynthia Shafer, Moraga, CA (US); Annette Walter, Mill Valley, CA (US); Mika K. Lindvall, Oakland, CA (US); Thomas Gesner, Kensington, CA (US); Laura Doyle, Oakland, CA (US)

Correspondence Address: CHIRON CORPORATION;INTELLECTUAL PROPERTY - R440, PO BOX 8097, EMERYVILLE, CA, 94662-8097, US

(73) Assignee: NOVARTIS VACCINES AND DIAGNOSTICS, INC., Emeryville, CA (US)

(21) Appl. No.: 11/735,302

(22) Filed: Apr. 13, 2007

Related U.S. Application Data
(60) Provisional application No. 60/793,691, filed on Apr. 19, 2006.

(51) Int. Cl. A61K 31/5375; A61K 31/44; A61K 31/445; A61P 35/00; C07D 239/20; C07D 295/00; C07D 241/02; C07D 213/02; A61K 31/496; A61K 31/506

(52) U.S. Cl. 514/234.5; 514/252.19; 514/253.09; 514/256; 514/318; 514/338; 544/123; 544/295; 544/316; 544/364; 546/193; 546/275.7

(57) ABSTRACT

New compounds capable of acting as CDC7 inhibitors are provided. The compounds are useful either alone or in combination with at least one additional therapeutic agent, in the prophylaxis or treatment of CDC7 mediated diseases, such as cancer. The compounds have the Formula (I) or (II), where the values of the variables are defined herein.

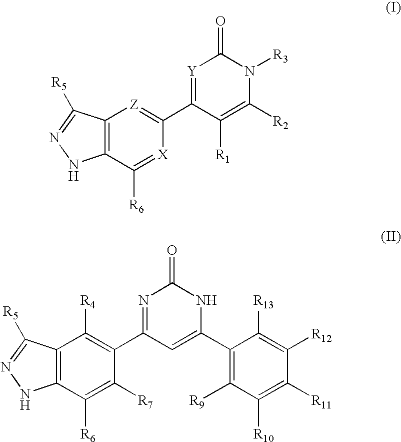

### CROSS-REFERENCE TO RELATED APPLICATION

This application claims the benefit of U.S. Provisional Application No. 60/793,691, filed Apr. 19, 2006.

### FIELD OF THE INVENTION

This invention relates to CDC7 inhibitors and provides new compounds, compositions of the new compounds together with pharmaceutically acceptable carriers, and uses of the new compounds, either alone or in combination with at least one additional therapeutic agent, in the prophylaxis or treatment of CDC7 mediated diseases, such as cancer.

### BACKGROUND

In eukaryotes, DNA replication is strictly regulated during the cell cycle and occurs only once and only during S phase (reviewed by Bell and Dutta, “DNA replication in eukaryotic cells” Annu Rev Biochem 71:333-74 (2002)). DNA replication is initiated by formation of a pre-replication complex (pre-RC) at origins of replication during G1. After complex formation the pre-RC is converted into an initiation complex by the concerted activity of two S-phase kinases, Cdk2/cyclinE and CDC7/Dbf4, also known as Hsk1 or CDC7L1. Hsk1 is the S. pombe CDC7 homolog. By searching EST databases for sequences similar to those of CDC7 and Hsk1, Jiang and Hunter identified a partial human CDC7 cDNA (Jiang and Hunter, “Identification and characterization of a human protein kinase related to budding yeast CDC7p” PNAS 23;94(26): 14320-5 (1997)). They used the partial cDNA to isolate a full-length cDNA from a HeLa cell library. The predicted 574-amino acid human CDC7 protein contains the 11 conserved subdomains found in all protein serine/threonine kinases as well as 3 additional sequences (kinase inserts) between subdomains I and II, VII and VIII, and X and XI. The kinase domains of human and S. cerevisiae CDC7 share 44% protein sequence identity. Human CDC7 has a molecular mass of 64 kD and is predominantly localized in the nucleus. Hess et al., “A human homolog of the yeast CDC7 gene is overexpressed in some tumors and transformed cell lines” Gene 211(1):133-40 (1998) reported that CDC7L1 was expressed in many normal tissues, but was overexpressed in all transformed cell lines tested and in certain tumor types.

CDC7, a serine/threonine kinase, plays an essential role in initiation of DNA replication in eukaryotic cells (Jiang et al., EMBO J 18:5703 (1999)). After assembly of the pre-replication complex to the replication origin, the CDC7 kinase phosphorylates MCM (minichromosome maintenance) proteins and allows for recruitment of CDC45 and DNA polymerase thereby initiating DNA replication (Kim et al., Mutation Research 532:29(2003)). CDC7 requires association with one of its cofactors, ASK (also known as DBF4) or ASKL1 (also known as Drf1), for kinase activation (Ogino et al., J Biol Chem 276:31376 (2001); Sato et al., Genes to Cells 8:451 (2003); Montagnoli et al., EMBO J 21:3171 (2002); Yoshizawa-Sugata et al., J Biol Chem 280, 13062 (2005)). Mice deficient for CDC7 die between day 3.5 and 6.5 indicating that CDC7 is essential for early embryonic development (Kim et al., EMBO J 21:2168 (2002)). Conditional knock-down of CDC7 in mouse ES cell lines (CDC7−/−tg) revealed immediate inhibition of cell proliferation, rapid cessation of DNA synthesis and arrest in S phase progression (Kim et al. (2002)). CDC7 has been implicated in DNA damage checkpoint signaling in response to Etoposide treatment or DNA single strand breaks (Costanzo et al., J Mol Cell 11:203 (2003)). A role for CDC7 in DNA damage response is supported by the observation that CDC7 depleted mouse ES cells accumulate RAD51 foci in the nucleus (Kim et al. (2002)). Deletion of CDC7 in yeast results in hypersensitivity to hydroxyurea treatment (Weinreich et al., EMBO J 18:5334 (1999)).

The serine/threonine kinase CDC7 plays an important role in the initiation of DNA replication and recently has been implicated in S phase checkpoint signaling(reviewed in Kim, Yamada and Masai, “Functions of mammalian CDC7 kinase in initiation/monitoring of DNA replication and development” Mutat Res 532(1-2):29-40 (2003)). The CDC7 kinase forms a complex with Dbf4, its regulatory subunit also known as ASK to generate an active Ser/Thr kinase. CDC7/Dbf4 kinase activity is required for initiation of DNA replication and subsequent transition into S-phase of the cell cycle. A second activator protein of CDC7 called Drf1 or ASKL1 has been identified in human cells, and appears to be involved in both S and M phase progression (Montagnoli et al., “Drf1, a novel regulatory subunit for human CDC7 kinase” EMBO J 21(12):3171-81 (2002); Yoshizawa-Sugata, “A second human Dbf4/ASK-related protein, Drf1/ASKL1, is required for efficient progression of S and M phases” Biol Chem 280(13):13062-70 (2005)). CDC7 knock-out mice are embryonic lethal between E3.5 and E6.5 (Kim et al., “Inactivation of CDC7 kinase in mouse ES cells results in S-phase arrest and p53-dependent cell death” EMBO J 21(9):2168-79 (2002)). However, the analysis of conditional CDC7 as well as conditional Dbf4 knock-out ES cell lines revealed the essential roles of both proteins in mammalian cell proliferation and DNA synthesis (Kim et al., “Hypomorphic mutation in an essential cell-cycle kinase causes growth retardation and impaired spermatogenesis” EMBO J 22(19):5260-72 (2003); Yamashita et al, “Functional analyses of mouse ASK, an activation subunit for CDC7 kinase, using conditional ASK knockout ES cells” Genes Cells 10(6):551-63 (2005)).

DNA replication starts by assembling a pre-replication complex (pre-RC) onto origins marked by a six-member origin recognition complex (ORC) during G1 phase of the cell cycle. Binding of Cdc6 and Cdt1 facilitates the loading of the minichromosome maintenance (MCM) complex onto the ORC. The MCM2-7 heterohexamer complex is considered to be a good candidate to function as the helicase that unwinds DNA ahead of the replication fork during S-phase although to date only the purified MCM467 complex has been demonstrated to have in vitro helicase activity (Lei et al., “Initiating DNA synthesis: from recruiting to activating the MCM complex” Cell Sci 114(Pt 8):1447-54 (2001); Schechter et al., “DNA unwinding is an Mcm complex-dependent and ATP hydrolysis-dependent process” J Biol Chem 279(44):45586-93 (2004)). MCM proteins are the major physiological substrates of CDC7. In S. cerevisiae a mutation in MCM5 bob-1 has been shown to bypass the requirement for CDC7/Dbf4 kinase activity (Hardy et al., “MCM5/cdc46-bob1 bypasses the requirement for the S phase activator CDC7p” PNAS 94(7):3151-5 (1997)). Among the six subunits that form the MCM2-7 complex, MCM2, MCM4 and MCM6 have been shown to be direct substrates of CDC7 in vitro and in cells. Two-dimensional tryptic radio-labeled phosphopeptide-mapping analysis of MCM2 phosphorylated by CDC7/Dbf4 revealed seven phosphorylation sites in vitro (Jiang et al., “Mammalian CDC7-Dbf4 protein kinase complex is essential for initiation of DNA replication” EMBO J 18(20):5703-13 1999). Recently CDC7 phosphorylation sites on MCM2 have been mapped to encompass the residues S40, S50 and S108 (Montagnoli et al., “Identification of Mcm2 phosphorylation sites by S-phase-regulating kinases” J Biol Chem 281(15):10281-90 (2006)). Additional residues, such as residue S53, have been identified to be phosphorylated by CDC7 in vitro and in vivo (Cho et al., “CDC7 kinase phosphorylates serine residues adjacent to acidic amino acids in the minichromosome maintenance 2 protein” PNAS 103(31):11521-6 (2006); Tsuji T et al., “Essential role of phosphorylation of MCM2 by CDC7/Dbf4 in the initiation of DNA replication in mammalian cells” Mol Biol Cell 17(10):4459-72 (2006)). Further, MCM2 can also be phosphorylated by another S-phase kinase, Cdk2/CycE, during DNA replication and by the ATM and ATM- and Rad3-related (ATR) checkpoint kinases in response to genotoxic stress (Cortez et al, “Minichromosome maintenance proteins are direct targets of the ATM and ATR checkpoint kinases” PNAS 101(27):10078-83 (2004); Yoo et al., “Mcm2 is a direct substrate of ATM and ATR during DNA damage and DNA replication checkpoint responses” J Biol Chem 279(51):53353-64 (2004)). Recently it has been reported that CDC7 mediates phosphorylation of MCM4 and MCM6 (Sheu and Stillman, “CDC7-Dbf4 phosphorylates MCM proteins via a docking site-mediated mechanism to promote S phase progression” Mol Cell 24(1):101-13 (2006); Masai H et al., “Phosphorylation of MCM4 by CDC7 kinase facilitates its interaction with Cdc45 on the chromatin” J Biol Chem 281(51):39249-61 (2006)). Although the functional relevance and redundancy between phosphorylation sites remains to be elucidated, phosphorylation of MCM proteins by CDC7 in general promotes S phase progression.

Recently, CDC7 has emerged as an attractive target for cancer therapy. Depletion of CDC7 using siRNA oligonucleotides results in induction of apoptosis in cancer cell lines while normal dermal fibroblast cells are spared) Montagnoli et al., Cancer Res 64, 7110 (2004)). Further, CDC7 mediated phosphorylation sites on MCM2, MCM4 and MCM6 in tumor cells have been identified, but the functional relevance of those sites remains to be determined (Montagnoli et al., J of Biol Chem 281:10281 (2006); Tsuji et al., Mol Biol Cell 17:4459-4472 (2006); Masai et al., J Biol Chem 281:39249-39261 (2006); Sheu et al., Mol Cell 24:101-113 (2006)). There is evidence that the CDC7/Dbf4 complex is a target of the S checkpoint response to genotoxic stress. In HU-treated S. cerevisiae, Rad53 phosphorylates Dbf4 resulting in a removal of the kinase complex from chromatin and in inhibition of CDC7/Dbf4 kinase activity. Deletion of CDC7 results in HU hypersensitivity (Weinreich M and Stillman B, 1999). Further, Xenopus egg extracts treated with Etoposide, a Topoisomerase II inhibitor used in the clinic as anti-cancer agent, resulted in activation of a DNA damage checkpoint that required ATR, blocking CDC7/Dbf4 kinase activity (Costanzo 2003). This is contrary to recent data indicating that the CDC7/Dbf4 kinase is active during replication stress and contributes to hyper-phosphorylation of MCM2 in response to HU and Etoposide treatment (Tenca P et al., 2007). Further depletion of CDC7 using siRNA in the presence of those drugs increased cell death.

Disease-related mutations in the tumor suppressor gene Men1 have been identified that block the interaction of menin with Dbf4, a cofactor required for CDC7 kinase activity, thereby contributing to the disease “Multiple endocrine neoplasia type I (MEN1) (Schnepp R W et al., 2004). Further more increased expression levels of CDC7 in breast cancer tissue samples, in particular ER and PR negative samples, have been detected based on in-house microarray analysis. This information could be used to identify a patient population susceptible to CDC7 inhibition.

Although the role of CDC7 in S-phase checkpoint regulation is not completely understood, there is evidence suggesting that a CDC7 inhibitor will show efficacy in cancer patients alone and as combination therapy with chemotherapeutic agents affecting DNA replication. However, there have been no specific CDC7 inhibitors to date approved for the treatment of cancer.

Accordingly, there is a need for potent and specific inhibitors of CDC7 that are low molecular weight small molecules, as well as methods for screening for such compounds. Methods of treating CDC7 mediated diseases, such as cancer are also particularly desirable.

### SUMMARY

The present invention provides potent and specific inhibitors of CDC7 that are low molecular weight small molecules. Thus, there has been provided, in accordance with one aspect of the invention, compounds of formula (I):

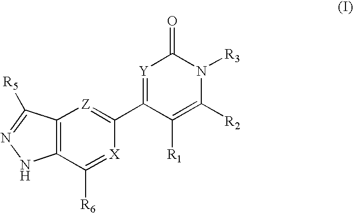

wherein X is N or CR₇;

Y is N or CR₈;

Z is N or CR₄;

R₁ is selected from the group consisting of H, halo, alkyl, substituted alkyl, hydroxy, alkoxy, substituted alkoxy, amino, and substituted amino;

R₂ is selected from the group consisting of alkyl, substituted alkyl, alkoxy, substituted alkoxy, amino, substituted amino, aryloxy, substituted aryloxy, heteroaryloxy, substituted heteroaryloxy, cycloalkyloxy, substituted cycloalkyloxy, heterocyclyloxy, substituted heterocyclyloxy, aryl, substituted aryl, heteroaryl, substituted heteroaryl, cycloalkyl, substituted cycloalkyl, heterocyclyl, and substituted heterocyclyl;

R₃ is H, alkyl, substituted alkyl, aryl or substituted aryl;

R₄, R₆, R₇ and R₈ are independently selected from the group consisting of H, halo, alkyl, substituted alkyl, hydroxy, alkoxy, substituted alkoxy, amino, and substituted amino;

R₅ is selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl; or

a stereoisomer, tautomer, or pharmaceutically acceptable salt thereof.

In other embodiments, new compounds are provided of Formula (II):

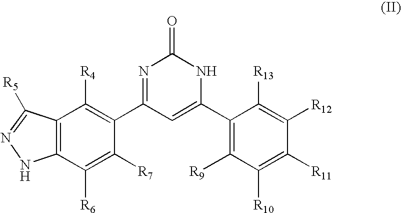

wherein R₄, R₆, and R₇ are independently selected from the group consisting of H, halo, alkyl, substituted alkyl, hydroxy, alkoxy, substituted alkoxy, amino, and substituted amino;

R₅ is selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl;

R₉, R₁₀, R₁₁, R₁₂, and R₁₃ are independently selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, substituted heterocyclyl, aryloxy, substituted aryloxy, heteroaryloxy, substituted heteroaryloxy, cycloalkyloxy, substituted cycloalkyloxy, heterocyclyloxy, and substituted heterocyclyloxy; or

a stereoisomer, tautomer, or pharmaceutically acceptable salt thereof.

In other aspects, the present invention provides methods for treating CDC7 related disorders in a human or animal subject in need of such treatment comprising administering to said subject an amount of a compound of formula (I) or (II) effective to inhibit CDC7 activity in the subject.

In other aspects, the CDC7 related disorder is cancer and the present invention provides methods for treating cancer in a human or animal subject in need of such treatment comprising administering to said subject an amount of a compound of formula (I) or (II) effective to reduce or prevent tumor growth in the subject. Representative cancers treatable in accordance with the invention include, but are not limited to, carcinoma such as bladder, breast, colon, kidney, liver, lung, including small cell lung cancer, esophagus, gallbladder, ovary, pancreas, stomach, cervix, thyroid, prostate, and skin carcinomas, including squamous cell carcinoma; hematopoietic tumors of lymphoid lineage, including leukemia, acute lymphocitic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xeroderma pigmentosum, keratoxanthoma, thyroid follicular cancer and Kaposi's sarcoma.

In yet other aspects, the present invention provides methods for treating CDC7 related disorders in a human or animal subject in need of such treatment comprising administering to said subject an amount of a compound of formula (I) or (II) effective to reduce or prevent tumor growth in the subject in combination with at least one additional agent for the treatment of cancer.

In yet other aspects, the present invention provides therapeutic compositions comprising at least one compound of formula (I) or (II) in combination with one or more additional agents for the treatment of cancer, as are commonly employed in cancer therapy.

In yet other aspects, the present invention provides a compound of formula (I) or (II) for use as a pharmaceutical. The present invention further provides for the use of a compound of formula (I) or (II) in the manufacture of a medicament for the treatment of cancer.

Another embodiment provides a method of screening for inhibition of CDC7 activity by a compound comprising exposing MCM2, CDC7 and ATP to the compound, and monitoring for phosphorylation of MCM2. In a more particular embodiment, the method comprises monitoring for phosphorylation of Ser108 on MCM2, as described in Example 80.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

The present invention relates to a novel class of small molecule CDC7 modulators. These compounds can be formulated into pharmaceutical compositions and are useful in inhibiting CDC7 in a human or animal subject, and in the treatment of CDC7 mediated diseases, such as cancer.

One embodiment of the invention provides for a new compounds comprising a substituted 4-(1H-indazol-5-yl)pyrimidin-2(1H)-one. In a more particular embodiment said 4-(1H-indazol-5-yl)pyrimidin-2(1H)-one is a substituted or unsubstituted 4-(1H-indazol-5-yl)-6-phenylpyrimidin-2(1H)-one. In another embodiment the compound has the formula (I) or (II). In a more particular embodiment the compound is a CDC7 inhibitor. In another embodiment thereof, the compound is a CDC7 inhibitor and is administered to a patient, more particularly, a patient with cancer, more particular still, a cancer comprising cells expressing CDC7.

Another embodiment of the invention provides new compounds of Formula (I):

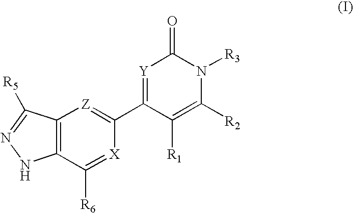

wherein X is N or CR₇;

Y is N or CR₈;

Z is N or CR₄;

R₁ is selected from the group consisting of H, halo, alkyl, substituted alkyl, hydroxy, alkoxy, substituted alkoxy, amino, and substituted amino;

R₂ is selected from the group consisting of alkyl, substituted alkyl, alkoxy, substituted alkoxy, amino, substituted amino, aryloxy, substituted aryloxy, heteroaryloxy, substituted heteroaryloxy, cycloalkyloxy, substituted cycloalkyloxy, heterocyclyloxy, substituted heterocyclyloxy, aryl, substituted aryl, heteroaryl, substituted heteroaryl, cycloalkyl, substituted cycloalkyl, heterocyclyl, and substituted heterocyclyl;

R₃ is H, alkyl, substituted alkyl, aryl or substituted aryl;

R₄, R₆, R₇ and R₈ are independently selected from the group consisting of H, halo, alkyl, substituted alkyl, hydroxy, alkoxy, substituted alkoxy, amino, and substituted amino;

R₅ is selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl; or

a stereoisomer, tautomer, or pharmaceutically acceptable salt thereof.

In a more particular embodiment, X is CR₇ and Z is CR₄. More particular still, R₄, R₆, and R₇ are H or halo. More particularly, R₄, R₆, and R₇ are H.

In another more particular embodiment, R₁ is H, halo or alkyl. More particularly, R₁ is H.

In another more particular embodiment, R₂ is aryl or substituted aryl. In another more particular embodiment, R₂ is heteroaryl or substituted heteroaryl. In another more particular embodiment, R₂ is cycloalkyl or substituted cycloalkyl. In another more particular embodiment, R₂ is heterocyclyl or substituted heterocyclyl. In another more particular embodiment, R₂ is phenyl or substituted phenyl.

In another more particular embodiment, R₃ is H or alkyl. More particularly, R₃ is methyl. More particularly R₃ is H.

In another more particular embodiment, R₅ is selected from the group consisting of H, halo, hydroxy, alkyl, substituted alkyl, amino, substituted amino, alkoxy, and substituted alkoxy. In another more particular embodiment, R₅ is H.

In another more particular embodiment, Y is N. In another more particular embodiment, Z is N. In another more particular embodiment, Y is CR₈ and only one of X and Z is N.

Another embodiment of the invention provides new compounds of Formula (II):

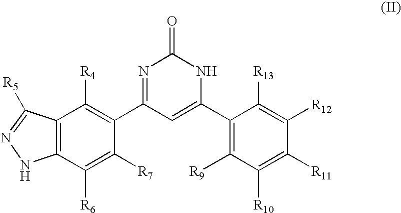

wherein R₄, R₆, and R₇ are independently selected from the group consisting of H, halo, alkyl, substituted alkyl, hydroxy, alkoxy, substituted alkoxy, amino, and substituted amino;

R₅ is selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl;

R₉, R₁₀, R₁₁, R₁₂, and R₁₃ are independently selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, substituted heterocyclyl, aryloxy, substituted aryloxy, heteroaryloxy, substituted heteroaryloxy, cycloalkyloxy, substituted cycloalkyloxy, heterocyclyloxy, and substituted heterocyclyloxy; or

a stereoisomer, tautomer, or pharmaceutically acceptable salt thereof.

In another more particular embodiment at least one of R₉, R₁₀, R₁₁, R₁₂, and R₁₃ is alkoxy. In another embodiment, at least one of R₉, R₁₀, R₁₁, R₁₂, and R₁₃ is halo, alkyl, or substituted alkyl.

In another more particular embodiment, R₁₁ is selected from the group consisting of halo, alkyl, substituted alkyl, alkoxy, substituted alkoxy, aryl, substituted aryl, heteroaryl, substituted heteroaryl, cycloalkyl, substituted cycloalkyl, heterocyclyl, substituted heterocyclyl, aryloxy, substituted aryloxy, heteroaryloxy, substituted heteroaryloxy, cycloalkyloxy, substituted cycloalkyloxy, heterocyclyloxy, and substituted heterocyclyloxy.

In another more particular embodiment, R₄, R₆, and R₇ are H or halo. More particular still, R₄, R₆, and R₇ are H.

In another more particular embodiment, R₅ is selected from the group consisting of H, halo, hydroxy, alkyl, substituted alkyl, amino, substituted amino, alkoxy, and substituted alkoxy. More particular still, R₅ is H.

In another more particular embodiment, the compound is selected from the group consisting of 6-(3-fluorophenyl)-4-(1H-indazol-5-yl)pyrimidin-2(1H)-one, 6-(2-fluoro-4-methoxyphenyl)-4-(1H-indazol-5-yl)pyrimidin-2(1H)-one, 6-(2,5-dimethoxy-phenyl)-4-(1H-indazol-5-yl)pyrimidin-2(1H)-one, 6-(3-fluoro-4-methoxyphenyl)-4-(1H-indazol-5-yl)pyrimidin-2(1H)-one, 6-(4-ethylphenyl)-4-(1H-indazol-5-yl)pyrimidin-2(1H)-one, 6-(3,4-dimethoxyphenyl)-4-(1H-indazol-5-yl)pyrimidin-2(1H)-one, 4-(1H-indazol-5-yl)-6-[3-(trifluoromethyl)phenyl]pyrimidin-2(1H)-one, 6-(2-fluorophenyl)-4-(1H-indazol-5-yl)pyrimidin-2(1H)-one, 6-(3-chlorophenyl)-4-(1H-indazol-5-yl)-pyrimidin-2(1H)-one, 4-(1H-indazol-5-yl)-6-phenylpyrimidin-2(1H)-one, 6-[3-(benzyloxy)phenyl]-4-(1H-indazol-5-yl)pyrimidin-2(1H)-one, 4-(1H-indazol-5-yl)-6-(4-morpholin-4-ylphenyl)pyrimidin-2(1H)-one, 4-(1H-indazol-5-yl)-6-(4-phenoxyphenyl)-pyrimidin-2(1H)-one, 6-[4-(benzyloxy)phenyl]-4-(1H-indazol-5-yl)pyrimidin-2(1H)-one, 4-(1H-indazol-5-yl)-6-(4-piperazin-1-ylphenyl)pyrimidin-2(1H)-one or a stereoisomer, tautomer, or pharmaceutically acceptable salt thereof.

Another embodiment of the present invention provides a pharmaceutical composition comprising a compound of formulas (I) or (II) and a pharmaceutically acceptable excipient or carrier.

Another embodiment of the present invention provides methods of treating human or animal subjects suffering from a cdc7 related disorder comprising administering to the subject an amount of a compound of the invention effective to inhibit CDC7 activity in the subject. In a more particular embodiment thereof, the cdc7 related disorder is a cancer disorder, and the invention provides methods of treating a human or animal subject in need of such treatment comprising administering to the subject a therapeutically effective amount of a compound of formula (I) or (II), either alone or in combination with other anticancer agents. In other aspects, the present invention provides methods for treating CDC7 related disorders in a human or animal subject in need of such treatment comprising administering to said subject an amount of a compound of formula (I) or (II) effective to reduce or prevent tumor growth in the subject. In yet other aspects, the present invention provides methods for treating CDC7 related disorders in a human or animal subject in need of such treatment comprising administering to said subject an amount of a compound of formula (I) or (II) effective to reduce or prevent tumor growth in the subject in combination with at least one additional agent for the treatment of cancer. A number of suitable anticancer agents to be used as combination therapeutics are contemplated for use in the methods of the present invention, as is hereinafter described in detail. More particular still, the cancer comprises cells that express CDC7.

Another embodiment of the present invention provides a method of inhibiting phosphorylation of MCM, more particularly MCM2, comprising exposing MCM or MCM2, CDC7 and ATP to a compound of any one of the previous embodiments. In a more particular embodiment, phosphorylation of Ser40 and/or Ser108 is inhibited on MCM2.

Another embodiment of the present invention provides use of a compound of formula (I) or (II) as a pharmaceutical, particularly for the treatment of cancer. In other embodiments, the present invention provides for the use of a compound of formula (I) or (II) in the manufacture of a medicament for the treatment of cancer.

Another embodiment of the present invention provides a method of screening for inhibition of CDC7 activity by a compound comprising exposing MCM2, CDC7 and ATP to a compound, and monitoring for phosphorylation of Ser108 on MCM2.

Another embodiment provides a method of identifying kinase activity of CDC7 comprising monitoring for phosphorylation of Ser108 on MCM2, wherein phosphorylation of Ser108 indicates activity of CDC7. A more particular embodiment provides further monitoring for phosphorylation of Ser40 on MCM2. In a more particular embodiment, said method of identifying activity of CDC7 is for the identification of an inhibitor of CDC7. In a more particular embodiment, said method of identifying activity of CDC7 is for identifying a patient in need of an inhibitor of CDC7. More particular still, said patient is suffering from cancer.

Another embodiment provides a method for screening for inhibitors of CDC7 comprising: exposing a potential inhibitor to CDC7 and MCM2 and monitoring for phosphorylation of Ser108 on MCM2, wherein said inhibitor of CDC7 is identified by reduced phosphorylation of Ser108 on MCM2. A more particular embodiment comprises exposing the potential inhibitor to CDC7, MCM2 and ATP. In a more particular embodiment said reduced phosphorylation of Ser108 on MCM2 is identified by reduced ATP depletion.

The present invention provides pharmaceutical compositions comprising at least one CDC7 inhibitor compound (e.g., a compound of formulas (I) or (II)) together with a pharmaceutically acceptable carrier suitable for administration to a human or animal subject, either alone or together with other anticancer agents.

In one embodiment, the present invention provides methods of treating human or animal subjects suffering from a cellular proliferative disease, such as cancer. Representative cancers treatable in accordance with the invention include, but are not limited to, carcinoma such as bladder, breast, colon, kidney, liver, lung, including small cell lung cancer, esophagus, gallbladder, ovary, pancreas, stomach, cervix, thyroid, prostate, and skin carcinomas, including squamous cell carcinoma; hematopoietic tumors of lymphoid lineage, including leukemia, acute lymphocitic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xeroderma pigmentosum, keratoxanthoma, thyroid follicular cancer and Kaposi's sarcoma. The present invention provides methods of treating a human or animal subject in need of such treatment, comprising administering to the subject a therapeutically effective amount of a CDC7 inhibitor compound of formulas (I) or (II), either alone or in combination with other anticancer agents.

In particular, compositions will either be formulated together as a combination therapeutic or administered separately. Anticancer agents for use with the invention include, but are not limited to, one or more of the following set forth below:

A. Kinase Inhibitors

Kinase inhibitors for use as anticancer agents in conjunction with the compositions of the present invention include inhibitors of Epidermal Growth Factor Receptor (EGFR) kinases such as small molecule quinazolines, for example gefitinib (U.S. Pat. No. 5,457,105, U.S. Pat. No. 5,616,582, and U.S. Pat. No. 5,770,599), ZD-6474 (WO 01/32651), erlotinib (Tarceva®, U.S. Pat. No. 5,747,498 and WO 96/30347), and lapatinib (U.S. Pat. No. 6,727,256 and WO 02/02552); Vascular Endothelial Growth Factor Receptor (VEGFR) kinase inhibitors, including SU-11248 (Sutent®, WO 01/60814), SU 5416 (U.S. Pat. No. 5,883,113 and WO 99/61422), SU 6668 (U.S. Pat. No. 5,883,113 and WO 99/61422), CHIR-258 (U.S. Pat. No. 6,605,617 and U.S. Pat. No. 6,774,237), vatalanib or PTK-787 (U.S. Pat. No. 6,258,812), VEGF-Trap (WO 02/57423), B43-Genistein (WO 09606116), fenretinide (retinoic acid p-hydroxyphenylamine) (U.S. Pat. No. 4,323,581), IM-862 (WO 02/62826), bevacizumab or Avastin® (WO 94/10202), KRN-951, 3-[5-(methylsulfonylpiperadine methyl)-indolyl]-quinolone, AG-13736 and AG-13925, pyrrolo[2,1-f][1,2,4]triazines, ZK-304709, Veglin®, VMDA-3601, EG-004, CEP-701 (U.S. Pat. No. 5,621,100), Cand5 (WO 04/09769); Erb2 tyrosine kinase inhibitors such as pertuzumab (WO 01/00245), trastuzumab, and rituximab; Akt protein kinase inhibitors, such as RX-0201; Protein Kinase C (PKC) inhibitors, such as LY-317615 (WO 95/17182), and perifosine (US 2003171303); Raf/Map/MEK/Ras kinase inhibitors including sorafenib (BAY 43-9006), ARQ-350RP, LErafAON, BMS-354825 AMG-548, and others disclosed in WO 03/82272; Fibroblast Growth Factor Receptor (FGFR) kinase inhibitors; Cell Dependent Kinase (CDK) inhibitors, including CYC-202 or roscovitine (WO 97/20842 and WO 99/02162); Platelet-Derived Growth Factor Receptor (PGFR) kinase inhibitors such as CHIR-258, 3G3 mAb, AG-13736, SU-11248 and SU6668; and Bcr-Abl kinase inhibitors and fusion proteins such as STI-571 or Gleevec® (imatinib).

B. Anti-Estrogens

Estrogen-targeting agents for use in anticancer therapy in conjunction with the compositions of the present invention include Selective Estrogen Receptor Modulators (SERMs) including tamoxifen, toremifene, raloxifene; aromatase inhibitors including Arimidex® or anastrozole; Estrogen Receptor Downregulators (ERDs) including Faslodex® or fulvestrant.

C. Anti-Androgens

Androgen-targeting agents for use in anticancer therapy in conjunction with the compositions of the present invention include flutamide, bicalutamide, finasteride, aminoglutethamide, ketoconazole, and corticosteroids.

D. Other Inhibitors

Other inhibitors for use as anticancer agents in conjunction with the compositions of the present invention include protein farnesyl transferase inhibitors including tipifamib or R-115777 (US 2003134846 and WO 97/21701), BMS-214662, AZD-3409, and FTI-277; topoisomerase inhibitors including merbarone and diflomotecan (BN-80915); mitotic kinesin spindle protein (KSP) inhibitors including SB-743921 and MKI-833; protease modulators such as bortezomib or Velcade® (U.S. Pat. No. 5,780,454), XL-784; and cyclooxygenase 2 (COX-2) inhibitors including non-steroidal antiinflammatory drugs I (NSAIDs).

E. Cancer Chemotherapeutic Drugs

Particular cancer chemotherapeutic agents for use as anticancer agents in conjunction with the compositions of the present invention include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®, US 2004073044), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

F. Alkylating Agents

Alkylating agents for use in conjunction with the compositions of the present invention for anticancer therapeutics include VNP-40101M or cloretizine, oxaliplatin (U.S. Pat. No. 4,169,846, WO 03/24978 and WO 03/04505), glufosfamide, mafosfamide, etopophos (U.S. Pat. No. 5,041,424), prednimustine; treosulfan; busulfan; irofluven (acylfulvene); penclomedine; pyrazoloacridine (PD-115934); O6-benzylguanine; decitabine (5-aza-2-deoxycytidine); brostallicin; mitomycin C (MitoExtra); TLK-286 (Telcyta®); temozolomide; trabectedin (U.S. Pat. No. 5,478,932); AP-5280 (Platinate formulation of Cisplatin); porfiromycin; and clearazide (meclorethamine).

G. Chelating Agents

Chelating agents for use in conjunction with the compositions of the present invention for anticancer therapeutics include tetrathiomolybdate (WO 01/60814); RP-697; Chimeric T84.66 (cT84.66); gadofosveset (Vasovist®); deferoxamine; and bleomycin optionally in combination with electroporation (EPT).

H. Biological Response Modifiers

Biological response modifiers, such as immune modulators, for use in conjunction with the compositions of the present invention for anticancer therapeutics include staurosprine and macrocyclic analogs thereof, including UCN-01, CEP-701 and midostaurin (see WO 02/30941, WO 97/07081, WO 89/07105, U.S. Pat. No. 5,621,100, W093/07153, WO 01/04125, WO 02/30941, WO 93/08809, WO 94/06799, WO 00/27422, WO 96/13506 and WO 88/07045); squalamine (WO 01/79255); DA-9601 (WO 98/04541 and U.S. Pat. No. 6,025,387); alemtuzumab; interferons (e.g. IFN-a, IFN-b etc.); interleukins, specifically IL-2 or aldesleukin as well as IL-1, IL-3, IL-4, IL-5, IL-6, IL-7, IL-8, IL-9, IL-10, IL-11, IL-12, and active biological variants thereof having amino acid sequences greater than 70% of the native human sequence; altretamine (Hexalen®); SU 101 or leflunomide (WO 04/06834 and U.S. Pat. No. 6,331,555); imidazoquinolines such as resiquimod and imiquimod (U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612); and SMIPs, including benzazoles, anthraquinones, thiosemicarbazones, and tryptanthrins (WO 04/87153, WO 04/64759, and WO 04/60308).

I. Cancer Vaccines:

Anticancer vaccines for use in conjunction with the compositions of the present invention include Avicine® (Tetrahedron Letters 26, 1974 2269-70); oregovomab (OvaRex®); Theratope® (STn-KLH); Melanoma Vaccines; GI-4000 series (GI-4014, GI-4015, and GI-4016), which are directed to five mutations in the Ras protein; GlioVax-1; MelaVax; Advexin® or INGN-201 (WO 95/12660); Sig/E7/LAMP-1, encoding HPV-16 E7; MAGE-3 Vaccine or M3TK (WO 94/05304); HER-2VAX; ACTIVE, which stimulates T-cells specific for tumors; GM-CSF cancer vaccine; and Listeria monocytogenes-based vaccines.

J. Antisense Therapy:

Anticancer agents for use in conjunction with the compositions of the present invention also include antisense compositions, such as AEG-35156 (GEM-640); AP-12009 and AP-11014 (TGF-beta2-specific antisense oligonucleotides); AVI-4126; AVI-4557; AVI-4472; oblimersen (Genasense®); JFS2; aprinocarsen (WO 97/29780); GTI-2040 (R2 ribonucleotide reductase mRNA antisense oligo) (WO 98/05769); GTI-2501 (WO 98/05769); liposome-encapsulated c-Raf antisense oligodeoxynucleotides (LErafAON) (WO 98/43095); and Sirna-027 (RNAi-based therapeutic targeting VEGFR-1 mRNA).

The compounds of the invention can also be combined in a pharmaceutical composition with bronchiodilatory or antihistamine drugs substances. Such bronchiodilatory drugs include anticholinergic or antimuscarinic agents, in particular ipratropium bromide, oxitropium bromide, and tiotropium bromide, and β-2-adrenoreceptor agonists such as salbutamol, terbutaline, salmeterol and, especially, formoterol. Co-therapeutic antihistamine drug substances include cetirizine hydrochloride, clemastine fumarate, promethazine, loratadine, desloratadine diphenhydramine and fexofenadine hydrochloride.

The compounds of the invention can also be combined in a pharmaceutical composition with compounds that are useful for the treatment of a thrombolytic disease, heart disease, stroke, etc., (e.g., aspirin, streptokinase, tissue plasminogen activator, urokinase, anticoagulants, antiplatelet drugs (e.g., PLAVIX; clopidogrel bisulfate), a statin (e.g., LIPITOR or Atorvastatin calcium), ZOCOR (Simvastatin), CRESTOR (Rosuvastatin), etc.), a Beta blocker (e.g., Atenolol), NORVASC (amlodipine besylate), and an ACE inhibitor (e.g., lisinopril).

The compounds of the invention can also be combined in a pharmaceutical composition with compounds that are useful for the treatment of antihypertension agents such as, ACE inhibitors, lipid lowering agents such as statins, LIPITOR (Atorvastatin calcium), calcium channel blockers such as NORVASC (amlodipine besylate). The compound s of the present invention may also be used in combination with fibrates, beta-blockers, NEPI inhibitors, Angiotensin-2 receptor antagonists and platelet aggregation inhibitors.

For the treatment of inflammatory diseases, including rheumatoid arthritis, the compounds of the invention may be combined with agents such as TNF-α inhibitors such as anti-TNF-α monoclonal antibodies (such as REMICADE, CDP-870) and D2E7 (HUMIRA) and TNF receptor immunoglobulin fusion molecules (such as ENBREL), IL-1 inhibitors, receptor antagonists or soluble IL-1Rα (e.g. KINERET or ICE inhibitors), nonsterodial anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX (celecoxib), PREXIGE (lumiracoxib)), metalloprotease inhibitors (preferably MMP-13 selective inhibitors), p2×7 inhibitors, α2δ inhibitors, NEUROTIN, pregabalin, low dose methotrexate, leflunomide, hydroxyxchloroquine, d-penicillamine, auranofin or parenteral or oral gold.

The compounds of the invention can also be used in combination with the existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, lumiracoxib and etoricoxib, analgesics and intraarticular therapies such as corticosteroids and hyaluronic acids such as hyalgan and synvisc.

The compounds of the invention may also be used in combination with antiviral agents such as Viracept, AZT, acyclovir and famciclovir, and antisepsis compounds such as Valant.

The compounds of the present invention may also be used in combination with CNS agents such as antidepressants (sertraline), anti-Parkinsonian drugs (such as deprenyl, L-dopa, Requip, Mirapex, MAOB inhibitors such as selegine and rasagiline, comP inhibitors, such as Tasmar, A-2 inhibitors, dopamine reuptake inhibitors, NMDA antagonists, Nicotine agonists, Dopamine agonists, and inhibitors of neuronal nitric oxide synthase), and anti-Alzheimer's drugs such as donepezil, tacrine, α2δ inhibitors, NEUROTIN, pregabalin, COX-2 inhibitors, propentofylline or metryfonate.

The compounds of the present invention may also be used in combination with osteoporosis agents such as EVISTA (raloxifene hydrochloride), droloxifene, lasofoxifene or fosomax and immunosuppressant agents such as FK-506 and rapamycin.

In another aspect of the invention, kits that include one or more compounds of the invention are provided. Representative kits include a CDC7 inhibitor compound of the invention (e.g., a compound of formulas (I)-(II)) and a package insert or other labeling including directions for treating a cellular proliferative disease by administering an CDC7 inhibitory amount of the compound.

Another aspect of the invention provides functionally important CDC7 phosphorylation sites on MCM2. Generally, a mechanism is provided by which CDC7-mediated phosphorylation of the MCM complex contributes to origin activation. Towards that goal, a detailed analysis of the specific sites on MCM2 phosphorylated by the CDC7/Dbf4 complex using peptide separation and tandem mass spectrometry was performed. An in vitro analysis was done in order to have enough peptides to yield a first pass “map” of putative specific phosphorylation sites. Subsequent verification showed that these same sites are phosphorylated in vivo using RNAi mediated knockdown of Dbf4 in A549 lung cancer cells. The in vitro to in vivo workflow and analysis methodology is sufficiently general so that other kinase substrates of interest may be mapped and validated.

Phosphorylation site mapping using proteomics and mass spectrometry continues to present a challenge due to the relative low abundance of phosphopeptides. Therefore many studies have focused on the enrichment of phosphopeptides using metal-chelation chromatography such as IMAC-Fe or IMAC-Ga. (Posewitz, Anal. Chem 71:2883-2892 (1999). However these methods suffer from poor capacity due to non-specific binding of acidic peptides. Typically, only the most abundant phosphopeptides are captured, even in “model” proteins such as casein or ovalbumin.

More recently Beausoleil et al., “Large-scale characterization of HeLa cell nuclear phosphoproteins” PNAS 101(33):12130-12135 (2004) described a novel method to enrich for phosphopeptides that relies on the charge differential between phosphorylated and unmodified tryptic peptides. Using strong cation exchange chromatography at low pH, phosphopeptides could be separated. The resulting fractions were then further separated on reverse phase LCMS. Using this approach, nuclear phosphoproteins in HeLa cells were characterized and were found 2,002 phosphorylation sites from 967 proteins. This large scale approach allowed the automated identification of five phosphorylation sites on MCM2 in HeLa cells.

A more particular aspect of the invention provides a detailed and complete characterization of the phosphorylation sites on a single protein using mass spectrometry, followed by Western blotting confirmation of the sites found. Therefore a low throughput method that employs offline reverse phase HPLC followed by MALDI-qTOF tandem mass spectrometry on each of the HPLC fractions was used (Krokhin et al., “MALDI QqTOF MS combined with off-line HPLC for characterization of protein primary structure and post-translational modifications” J Biomol Tech 16(4)429-440 (2005)). Enrichment of phosphopeptides is not required, therefore peptides are not specifically excluded from analysis. Using this methodology, the identification of phosphorylation sites on MCM2 in vitro and in vivo that are specifically mediated by the CDC7/Dbf4 kinase complex are described. Nearly 75% sequence coverage of in vivo full-length immunopurified MCM2 was obtained. In addition to the sites previously found by other studies, a new site mediated by CDC7/Dbf4 was identified (S108). This site was previously found to be phosphorylated by ATR in response to DNA damage. However, our findings demonstrate that in the absence of exogenous DNA damage, S108 on MCM2 is phosphorylated by the CDC7/Dbf4 heterodimer.

The following definitions of terms are used throughout this specification and claims.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR₁₀R₁₁ where R₁₀ and R₁₁ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR₁₀R₁₁ where R₁₀ and R₁₁ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonylamino” refers to the group —NRC(O)NR₁₀R₁₁ where R is hydrogen or alkyl and R₁₀ and R₁₁ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NRC(S)NR₁₀R₁₁ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR₁₀R₁₁ where R₁₀ and R₁₁ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR₁₀R₁₁ where R₁₀ and R₁₁ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR₁₀R₁₁ where R₁₀ and R₁₁ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonylamino” refers to the group —NR—SO₂NR₁₀R₁₁ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkyenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkyenyl, aryl, substituted aryl, heteroaryl, substituted heteroaryl, heterocyclic and substituted heterocyclic are as defined herein.

“Amidino” refers to the group —C(═NR₁₂)R₁₀R₁₁ where R₁₀, R₁₁, and R₁₂ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R₁₀ and R₁₁ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Alkenyl” refers to alkenyl groups having from 2 to 6 carbon atoms and preferably 2 to 4 carbon atoms and having at least 1 and preferably from 1 to 2 sites of alkenyl unsaturation. Such groups are exemplified, for example, by vinyl, allyl, and but-3-en-1-yl.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to a vinyl (unsaturated) carbon atom.

“Alkynyl” refers to alkynyl groups having from 2 to 6 carbon atoms and preferably 2 to 3 carbon atoms and having at least 1 and preferably from 1 to 2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to an acetylenic carbon atom.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, substituted —NR—C(O)O-alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“CDC7 inhibitor” is used herein to refer to a compound that exhibits an IC₅₀ with respect to CDC7 activity of no more than about 100 μM and more typically not more than about 50 μM, as measured in the in vitro assay of CDC7/DBF4 inhibition, as described in Example 79, herein below. “IC₅₀” is that concentration of inhibitor which reduces the activity of an enzyme (e.g., Raf kinase) to half-maximal level. Representative compounds of the present invention have been discovered to exhibit inhibitory activity against CDC7. Compounds of the present invention preferably exhibit an IC₅₀ with respect to CDC7 of no more than about 10 μM, more preferably, no more than about 5 μM, even more preferably not more than about 1 μM, and most preferably, not more than about 200 nM, as measured in the CDC7 assays described herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR₁₃C(═NR₁₃)N(R₁₃)₂ where each R₁₃ is independently selected from the group consisting of hydrogen, alkyl, substituted alkyl, aryl, substituted aryl, heteroaryl, substituted heteroaryl, heterocyclic, and substituted heterocyclic and two R₁₃ groups attached to a common guanidino nitrogen atom are optionally joined together with the nitrogen bound thereto to form a heterocyclic or substituted heterocyclic group, provided that at least one R₁₃ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or unsaturated group having a single ring or multiple condensed rings, including fused bridged and spiro ring systems, from 1 to 10 carbon atoms and from 1 to 4 hetero atoms selected from the group consisting of nitrogen, sulfur or oxygen within the ring wherein, in fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydro-benzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O—).

“Spirocyclyl” refers to divalent saturated cyclic group from 3 to 10 carbon atoms having a cycloalkyl or heterocyclyl ring with a spiro union (the union formed by a single atom which is the only common member of the rings) as exemplified by the following structure:

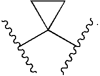

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Substituted sulfonyl includes groups such as methyl-SO₂—, phenyl—SO₂—, and 4-methylphenyl—SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl,—OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Homologue” refers to a sequence having at least 50% homology, or at least 60% homology, or at least 70% homology, or at least 80% homology, or at least 85% homology, or at least 90% homology, or at least 95% homology, or at least 96% homology, or at least 97% homology, or at least 98% homology, or at least 99% homology to the referenced sequence.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

The compounds of the invention are useful in vitro or in vivo in inhibiting the growth of cancer cells. The compounds may be used alone or in compositions together with a pharmaceutically acceptable carrier or excipient. Pharmaceutical compositions of the present invention comprise a therapeutically effective amount of a CDC7 inhibitor compound described herein formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds of the present invention may be administered to humans and other animals orally, parenterally, sublingually, by aerosolization or inhalation spray, rectally, intracistemally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrastemal injection, or infusion techniques.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass median aerodynamic diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

Effective amounts of the compounds of the invention generally include any amount sufficient to detectably inhibit CDC7 activity by any of the assays described herein, by other CDC7 activity assays known to those having ordinary skill in the art, or by detecting an inhibition or alleviation of symptoms of cancer. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

According to the methods of treatment of the present invention, tumor growth is reduced or prevented in a patient such as a human or lower mammal by administering to the patient a therapeutically effective amount of a compound of the invention, in such amounts and for such time as is necessary to achieve the desired result. By a “therapeutically effective amount” of a compound of the invention is meant a sufficient amount of the compound to treat tumor growth, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose. In general, treatment regimens according to the present invention comprise administration to a patient in need of such treatment from about 10 mg to about 2000 mg of the compound(s) of this invention per day in single or multiple doses.

In another aspect of the invention, kits that include one or more compounds of the invention are provided. Representative kits include a CDC7 inhibitor compound of formulas (I) or (II) and a package insert or other labeling including directions for treating a cellular proliferative disease by administering an CDC7 inhibitory amount of the compound.

The term “kit” as used herein comprises a container for containing the pharmaceutical compositions and may also include divided containers such as a divided bottle or a divided foil packet. The container can be in any conventional shape or form as known in the art which is made of a pharmaceutically acceptable material, for example a paper or cardboard box, a glass or plastic bottle or jar, a resealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

The kits of the present invention may also comprise, in addition to a CDC7 inhibitor, one or more additional pharmaceutically active compounds. Preferably, the additional compound is another anticancer agent described above in one of groups A-J. The additional compounds may be administered in the same dosage form as the CDC7 inhibitor or in different dosage forms. Likewise, the additional compounds can be administered at the same time as the CDC7 inhibitor or at different times.

The present invention will be understood more readily by reference to the following examples, which are provided by way of illustration and are not intended to be limiting of the present invention.

### EXAMPLES

Referring to the examples that follow, compounds of the present invention were synthesized using the methods described herein, or other methods, which are known in the art.

Mass spectrometric analysis was performed on one of two LCMS instruments: a Waters System (Alliance HT HPLC and a Micromass ZQ mass spectrometer; Column: Eclipse XDB-C18, 2.1×50 mm; solvent system: 5-95% (or 35-95%, or 65-95% or 95-95%) acetonitrile in water with 0.05% TFA; flow rate 0.8 mL/min; molecular weight range 200-1500; cone Voltage 20 V; column temperature 40° C.) or a Hewlett Packard System (Series 1100 HPLC; Column: Eclipse XDB-C18, 2.1×50 mm; solvent system: 1-95% acetonitrile in water with 0.05% TFA; flow rate 0.8 mL/min; molecular weight range 150-850; cone Voltage 50 V; column temperature 30° C.). All masses were reported as those of the protonated parent ions.

GCMS analysis is performed on a Hewlett Packard instrument (HP6890 Series gas chromatograph with a Mass Selective Detector 5973; injector volume: 1 μL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis is performed on the compounds with a Varian 300 MHz NMR (Palo Alto, Calif., USA). The spectral reference is either TMS or the known chemical shift of the solvent. Some compound samples are run at elevated temperatures (e.g., 75° C.) to promote increased sample solubility.

The purity of some of the invention compounds is assessed by elemental analysis (Desert Analytics, Tucson, Ariz., USA).

Melting points are determined on a Laboratory Devices Mel-Temp apparatus (Holliston, Mass., USA).

Preparative separations are carried out using a Flash 40 chromatography system and KP—Sil, 60A (Biotage, Charlottesville, Va., USA), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the invention may exhibit the phenomenon of tautomerism. As the chemical structures within this specification can only represent one of the possible tautomeric forms, it should be understood that the invention encompasses any tautomeric form of the drawn structure.

It is understood that the invention is not limited to the embodiments set forth herein for illustration, but embraces all such forms thereof as come within the scope of the above disclosure.

A modification of the chalcone synthesis can be carried out via the dibromochalcone as follows in Scheme 1 (Dora et al., Journal of Heterocyclic Chemistry 20:691-696 (1983)).

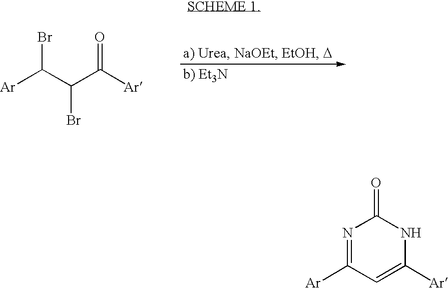

In addition to synthetic methods for pyrimidinones that utilize chalcones or Biginelli type reactions, pyrimidinones can also be synthesized from acetylenes and urea as shown in Scheme 2 (Sasakura et al., Synthetic Communications 18:259-264 (1988), Lee et al., Tetrahedron 61:8705-8710 (2005); Dora et al., Journal of Heterocyclic Chemistry 20:691-696 (1983) and Baddar et al., Journal of Heterocyclic Chemistry 13:257-268 (1976)).

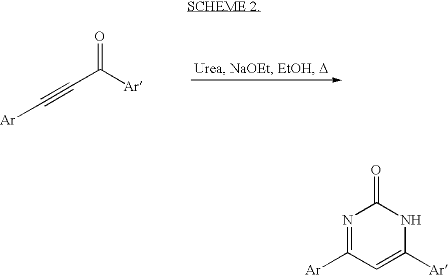

4-Aryl-6-alkylpyrimidinones can be synthesized from diones by the similar methodology used to synthesize the 4,6-diarylpyrimidinones (Scheme 3, Walker et al. WO 2003037896 (2003) p. 116; Carter et al. U.S. Pat. No. 6,780,870 (2004) p 14; Cai et al. WO 05121106 (2005) p. 71; and Abdel-Rahman et al., Egyptian Journal of Chemistry 30:231-238 (1989)).

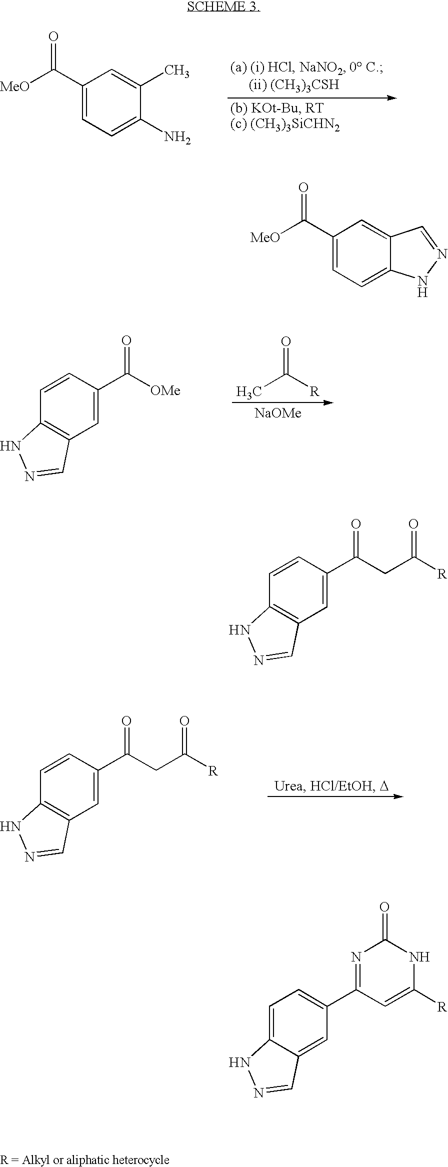

Aromatic heterocycles are incorporated by combining a heteroaromatic methylketones and the indazole aldehyde using standard acid conditions (urea, HCl, i-PrOH, Scheme 4(a), Bhendkar et al., W. Oriental Journal of Chemistry 19:731-732 (2003)). Other methodologies also exist for incorporation of five-membered ring heterocycles (Scheme 4(b) and 4(c), Babu et al., Indian Journal of Pharmaceutical Sciences 66:647-652 (2004)).

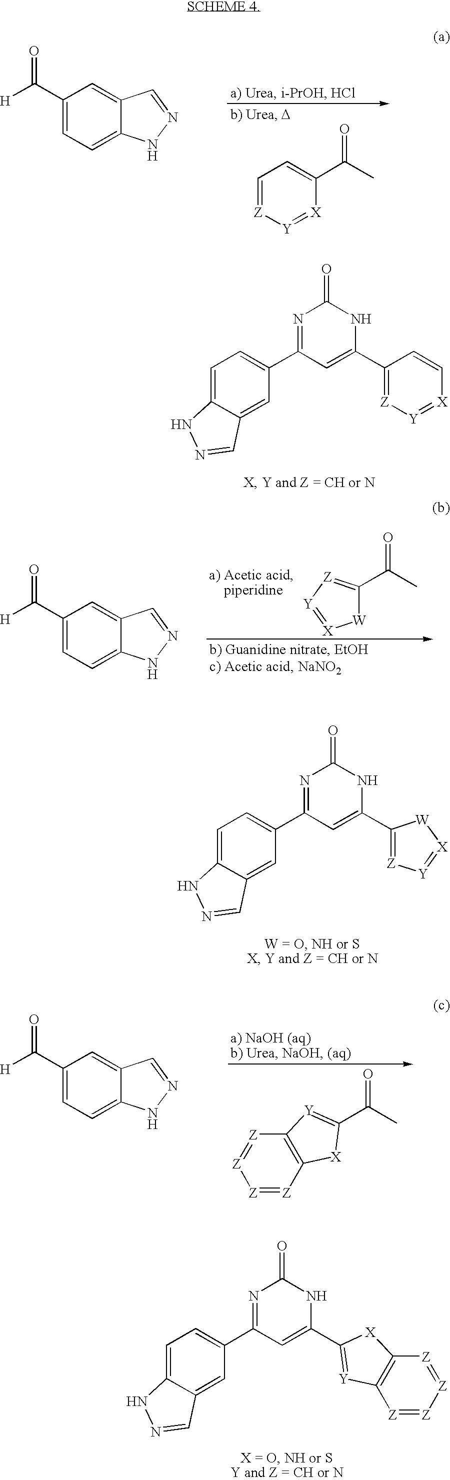

N-aryl 4,6-dialkylpyrimidinones, N-alkyl 4-alkyl-6-arylpyrimidinones and N-alkyl 6-alkyl-4-arylpyrimidinones can also be synthesized from N-aryl or N-alkylurea and the corresponding dione as shown in Scheme 5 (George et al., New Journal of Chemistry 27:568-576 (2003)).

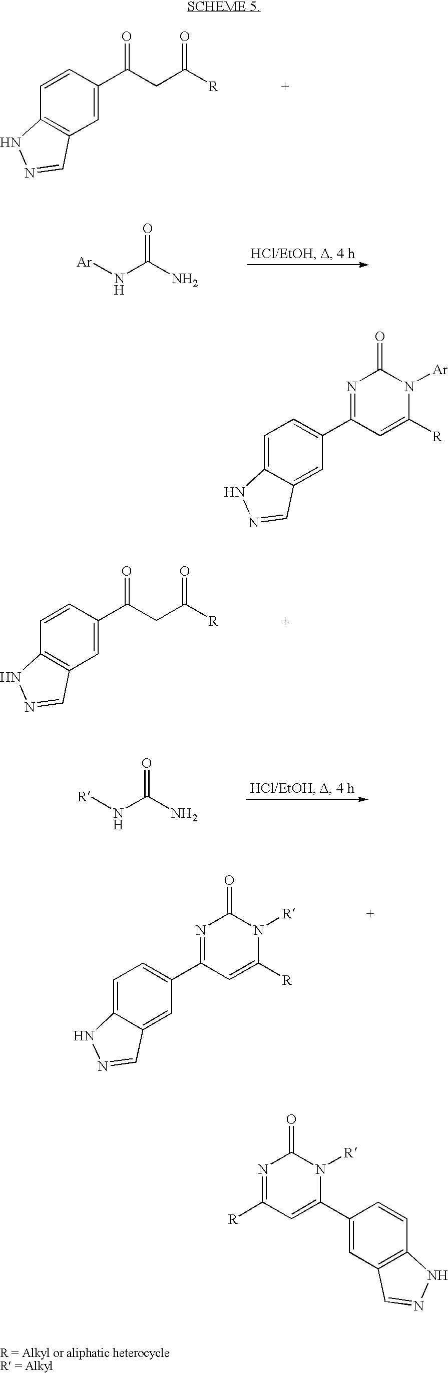

Small alkyl groups at C-5 of the pyrimidinone can be introduced by deprotonation and alkylation of a dione (Scheme 6, Cai WO05121106 (2005), p. 71) or by Wittig reaction (Scheme 7, Marzinzik et al., Journal of Organic Chemistry 63:723-727 (1998)) to give the corresponding chalcone which can be further functionalized to form the desired pyrimidinone.

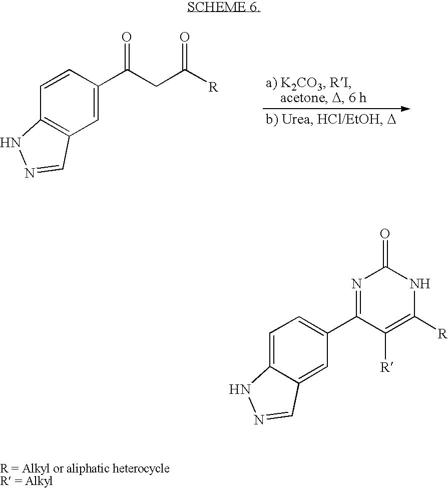

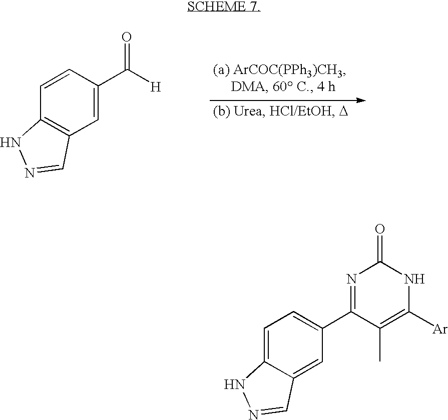

Several methods have been developed which incorporate aryl, alkyl and heteroaryl substituents at C-4 and C-6 and methyl at C-3 of the pyridone. Scheme 8 exemplifies these methods (8(a) Katritzky et al., Journal of Organic Chemistry 62:6210-6214 (1997); 8(b) Wang et al., Synthesis 487-490 (2003)).

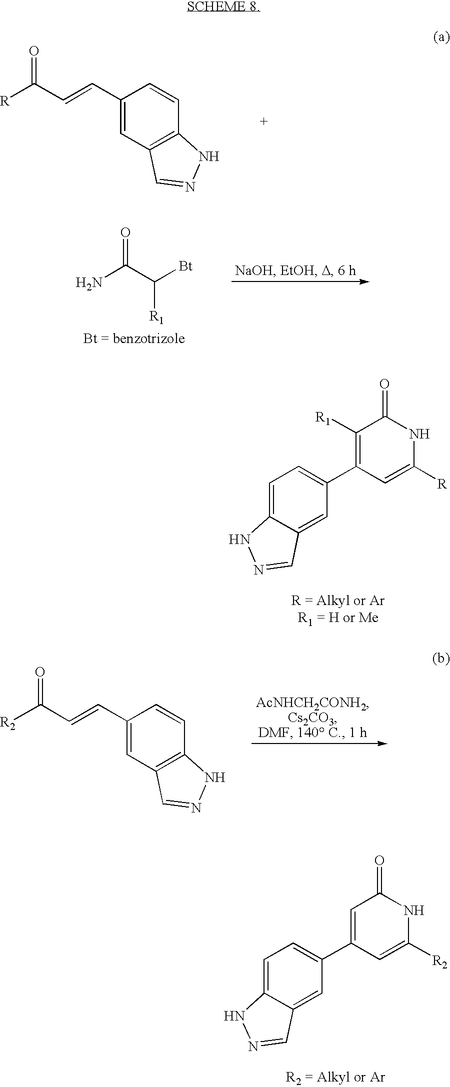

Furthermore, N-alkylated pyridones and pyridones substituted at C3 or C5 are accessible via the aminoazabutadiene chemistry shown in Scheme 9 (Hoberg et al., Synthesis No. 3, 142-144 (1970), Wittig et al., Justus Liebigs Annalen der Chemie 1075-1081 (1973), Barluenga et al., Tetrahedron Letters 29:4855-4858 (1988)).

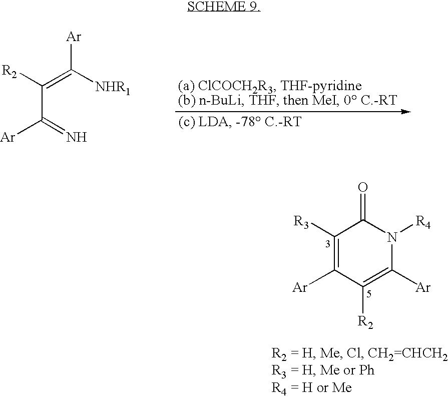

Azaindazole (or 1H-pyrazolopyridine) analogs can be made by synthesizing the requisite 5-bromoazaindazoles from the bromomethylnitropyridines (Scheme 10, Xie et al. WO 05092890 (2005) p. 300). Once the bromoazaindazoles are synthesized, the synthetic methodology is identical to that of the 4-indazole-6-arylpyrimdinone series.

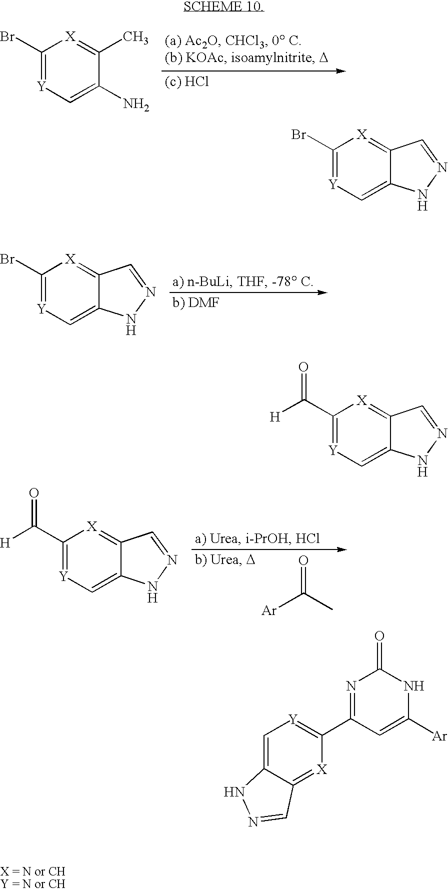

The C-3 position of the indazole can be substituted with alkyl groups as indicated in Scheme 11 (Li et al. US 2003/0199511 (2003), p. 120). In addition to methyl, other Grignard reagents could be used to incorporate other alkyl and aryl groups at C-3 of the indazole such as ethyl, propyl, iso-propyl, phenyl and substituted alkyl and aryl groups.

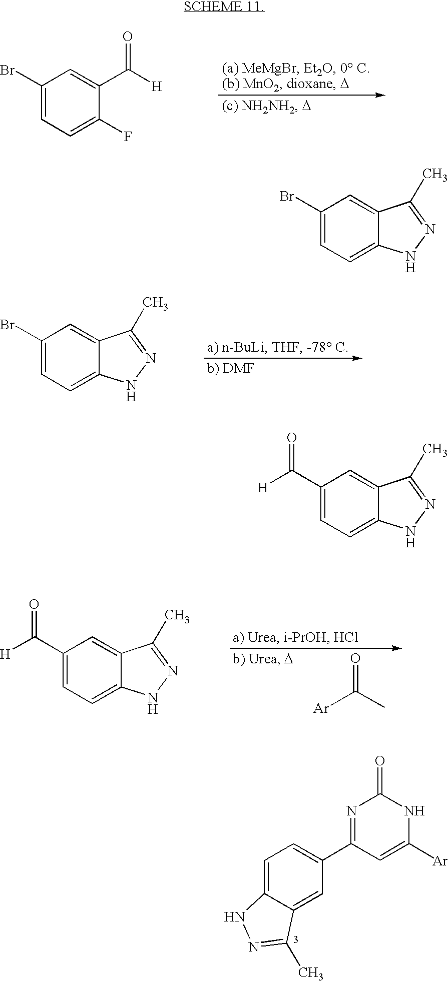

Other substituents at C-3 can also be incorporated such as methoxy, aliphatic heterocycles such as piperidine and methylene linked heterocycles such as morpholine (Scheme 12, Allen et al. WO 9749698 (1997) p. 84).

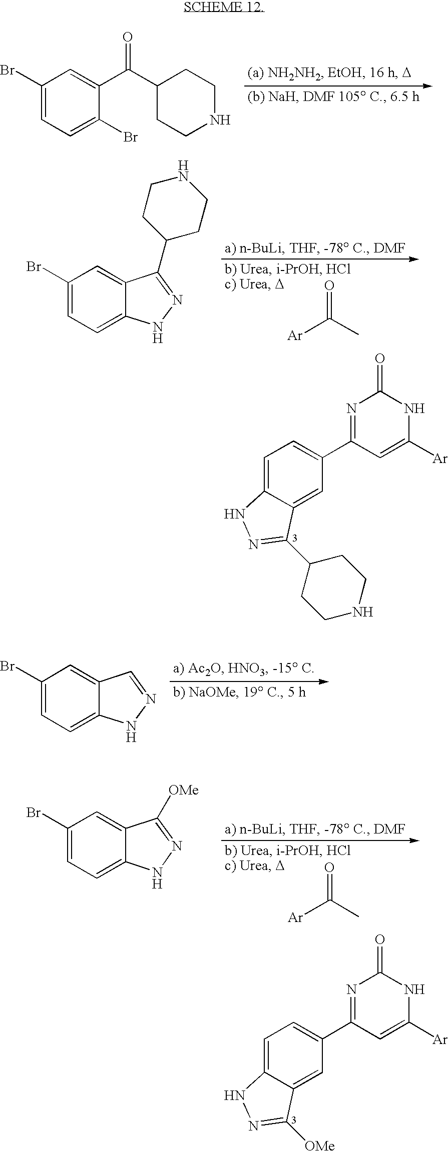

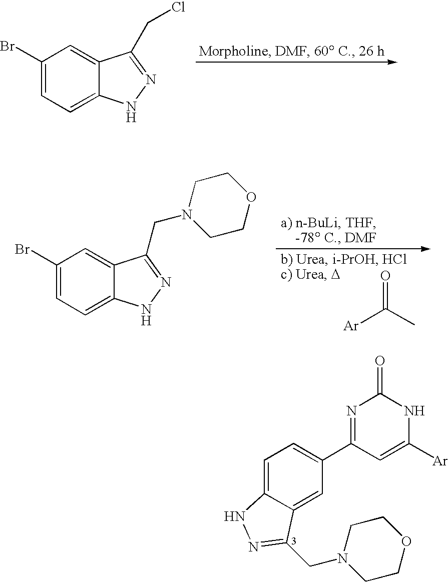

Longer chain aliphatic groups can also be incorporated at C-3 of the indazole as highlighted in Scheme 13 (Sasakura et al., Synthetic Communications 18:259-264 (1988)).

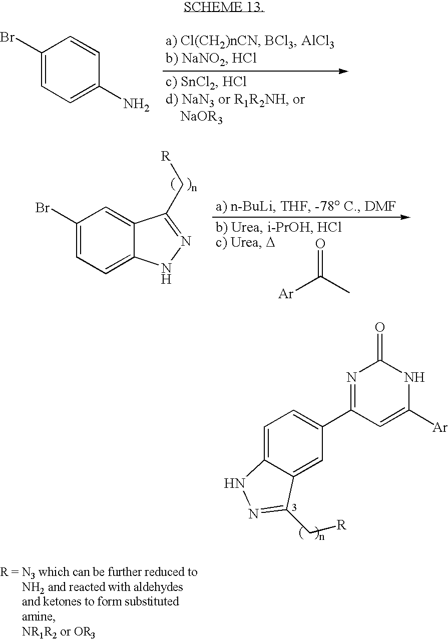

### Example 1

### Synthesis of 4-(1H-indazol-5-yl)-6-(4-phenoxyphenyl)pyrimidin-2(1H)-one

STEP 1:

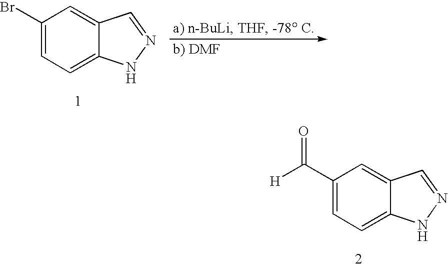

1H-indazole-5-carbaldehyde (2). n-Butyllithium (35.0 mL, 87.5 mmol) was added slowly to 5-bromoindazole (1, 4.98 g, 25.3 mmol) in THF (60 mL) at −78° C. After 30 min, the solution was warmed to −40° C. over 30 min and then cooled to −78° C. DMF (3.1 mL, 77.5 mmol) was added. After 15 min, the reaction flask was removed from the dry ice/acetone bath and stirred at room temperature for 2.5 h. The solution was quenched with H₂O. The aqueous layer was extracted with EtOAc. The organic layer was washed with H₂O and brine, dried over Na₂SO₄, filtered and concentrated to a golden oil. The crude material was purified by column chromatography (0-100% EtOAc/hexanes) to give 2 as a light yellow solid (1.91 g, 52% yield). LCMS m/z 147.0 (MH⁺), Rₜ 1.53 min.

Reference for the synthesis of 1H-indazole-5-carbaldehyde: E. Piatnitski, WO 2005/000813 p 37.
STEP 2:

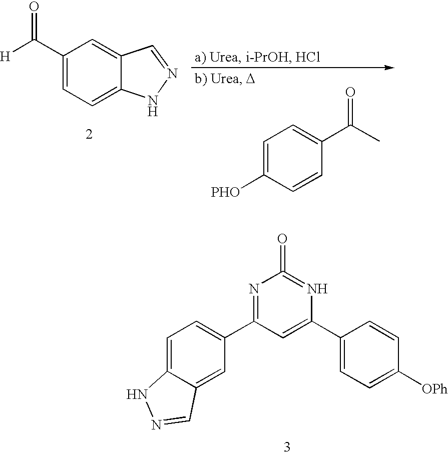

4-(1H-indazol-5-yl)-6-(4-phenoxyphenyl)pyrimidin-2(1H)-one (3). 1H-Indazole-5-carbaldehyde (2, 0.27 g, 1.85 mmol) and urea (0.33 g, 5.45 mmol) were stirred overnight at room temperature in i-PrOH (18 mL) and HCl (conc., 1.8 mL). At that time, the viscous solution was divided into nine equal portions. To one portion was added 4′-phenoxyacetophenone (0.0531 g, 0.25 mmol) and additional urea. The reaction was heated at 80° C. overnight in a sealed vial. The reaction mixture was then cooled, concentrated and purified by reverse phase HPLC to give 3 as the TFA salt (9.6 mg, 99% purity). LCMS m/z 381.1 (MH⁺), Rₜ 2.39 min.

Reference for acid-catalyzed Biginelli: Sedova et al., Chem. Heterocyclic Compounds 40(2):194-202 (2004).

### Examples 2-16

The compounds in the following Table 1 were synthesized using the foregoing methods and procedures, and were named using ACD Name for ChemSketch version 10.00 software (Aug. 31, 2006) available from Advanced Chemistry Development, Inc., 110 Yonge Street 14th Floor, Toronto, Ontario, Canada.

TABLE 1

| | | | LC/MS |
|---|---|---|---|
| Example | Structure | Name | (m/z, Rt) |
| 2 | 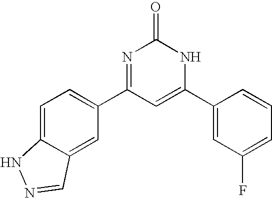 | 6-(3-fluorophenyl)-4-(1H- indazol-5-yl)pyrimidin-2(1H)- one | 307.0, 2.00 |
| 3 | 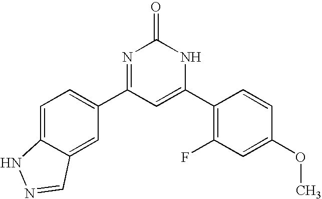 | 6-(2-fluoro-4-methoxyphenyl)- 4-(1H-indazol-5-yl)pyrimidin- 2(1H)-one | 337.0, 1.98 |
| 4 | 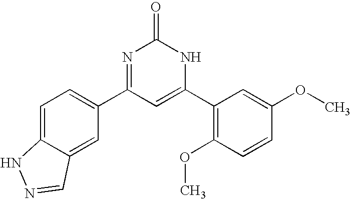 | 6-(2,5-dimethoxyphenyl)-4-(1H- indazol-5-yl)pyrimidin-2(1H)- one | 349.0, 2.00 |
| 5 | 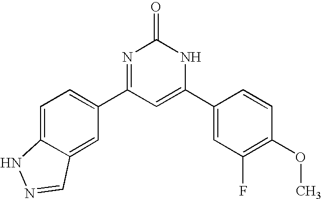 | 6-(3-fluoro-4-methoxyphenyl)- 4-(1H-indazol-5-yl)pyrimidin- 2(1H)-one | 337.0, 1.98 |
| 6 | 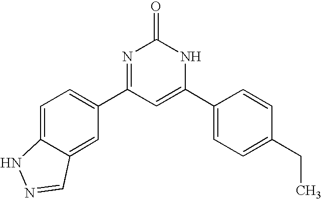 | 6-(4-ethylphenyl)-4-(1H- indazol-5-yl)pyrimidin-2(1H)- one | 317.1, 2.16 |
| 7 | 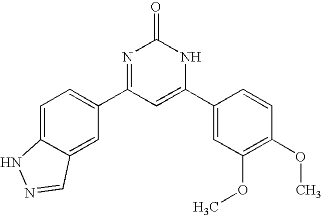 | 6-(3,4-dimethoxyphenyl)-4-(1H- indazol-5-yl)pyrimidin-2(1H)- one | 349.1, 1.89 |
| 8 | 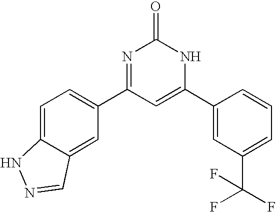 | 4-(1H-indazol-5-yl)-6-[3-(tri- fluoromethyl)phenyl]pyrimidin- 2(1H)-one | 357.0, 2.29 |
| 9 | 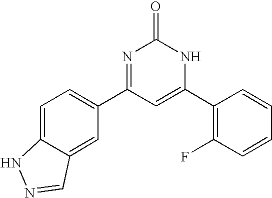 | 6-(2-fluorophenyl)-4-(1H- indazol-5-yl)pyrimidin-2(1H)- one | 307.0, 1.92 |
| 10 | 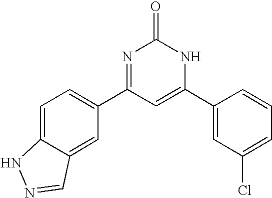 | 6-(3-chlorophenyl)-4-(1H- indazol-5-yl)pyrimidin-2(1H)- one | 323.0, 2.15 |
| 11 | 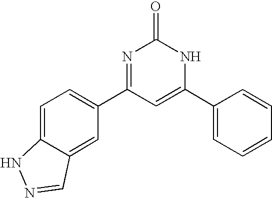 | 4-(1H-indazol-5-yl)-6-phenyl- pyrimidin-2(1H)-one | 289.0, 1.85 |
| 12 | 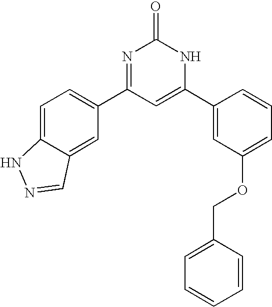 | 6-[3-(benzyloxy)phenyl]-4-(1H- indazol-5-yl)pyrimidin-2(1H)- one | 395.0, 2.48 |
| 13 | 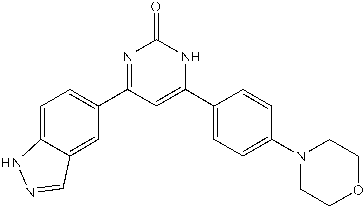 | 4-(1H-indazol-5-yl)-6-(4- morpholin-4-ylphenyl)- pyrimidin-2(1H)-one | 374.1, 1.92 |
| 14 | 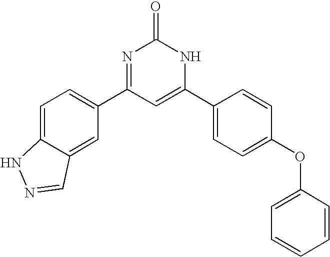 | 4-(1H-indazol-5-yl)-6-(4- phenoxyphenyl)pyrimidin- 2(1H)-one | 381.1, 2.39 |
| 15 | 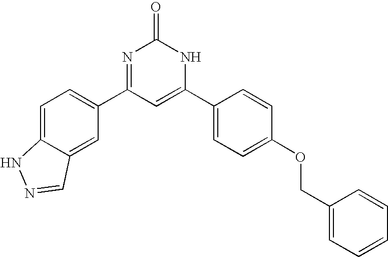 | 6-[4-(benzyloxy)phenyl]-4-(1H- indazol-5-yl)pyrimidin-2(1H)- one | 395.1, 2.44 |
| 16 | 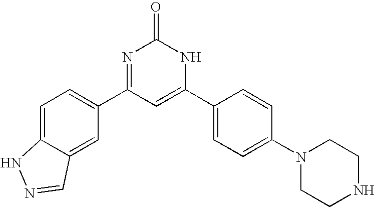 | 4-(1H-indazol-5-yl)-6-(4- piperazin-1-ylphenyl)pyrimidin- 2(1H)-one | 373.0, 1.53 |

The compounds in Table 1 were synthesized according to the Examples provided above. CDC7 inhibitory (IC₅₀) values of the compounds were determined according to Biological Method 1.

As described in Example 83 (in vitro assay of CDC7/DBF4 inhibition), each of the compounds of Table 1 exhibited an IC₅₀ value of less than 1 μM with respect to inhibition of CDC7/DBF4. Many of the Examples of Table 1 exhibited IC₅₀ values of less than 0.1 μM and even less than 0.01 μM with respect to inhibition of CDC7. For this reason, each of the compounds are individually preferred and are preferred as a member of a group.

### Examples 17-80

The compounds in the following Table 2 can be synthesized using the foregoing methods and procedures, and are named using ACD Name for ChemSketch version 10.00 software (Aug. 31, 2006) available from Advanced Chemistry Development, Inc., 110 Yonge Street 14th Floor, Toronto, Ontario, Canada.

TABLE 2

| Example | Structure | Name | MW |
|---|---|---|---|
| 17 | 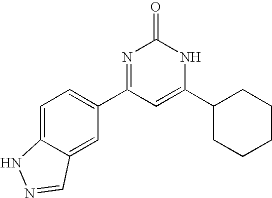 | 6-cyclohexyl-4-(1H-indazol-5- yl)pyrimidin-2(1H)-one | 294.4 |
| 18 | 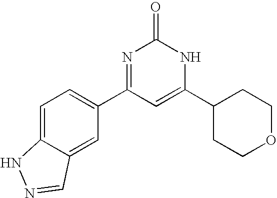 | 4-(1H-indazol-5-yl)-6- (tetrahydro-2H-pyran-4- yl)pyrimidin-2(1H)-one | 296.3 |
| 19 | 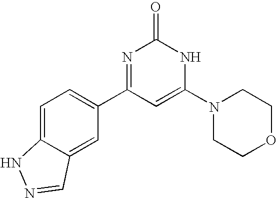 | 4-(1H-indazol-5-yl)-6- morpholin-4-ylpyrimidin- 2(1H)-one | 297.3 |
| 20 | 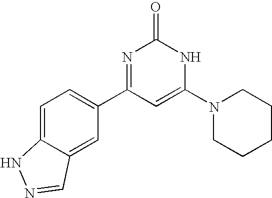 | 4-(1H-indazol-5-yl)-6- piperadin-1-ylpyrimidin-2(1H)- one | 295.3 |
| 21 | 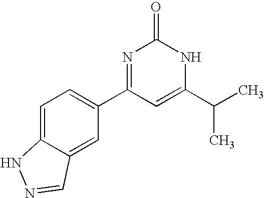 | 4-(1H-indazol-5-yl)-6- isopropylpyrimidin-2(1H)-one | 254.3 |
| 22 | 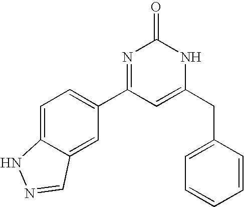 | 6-benzyl-4-(1H-indazol-5- yl)pyrimidin-2(1H)-one | 302.3 |
| 23 | 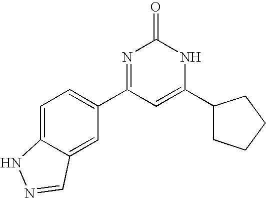 | 6-cyclopentyl-4-(1H-indazol-5- yl)pyrimidin-2(1H)-one | 280.3 |
| 24 | 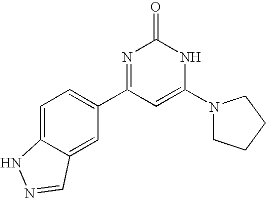 | 4-(1H-indazol-5-yl)-6- pyrrolidin-1-ylpyrimidin-2(1H)- one | 281.3 |
| 25 | 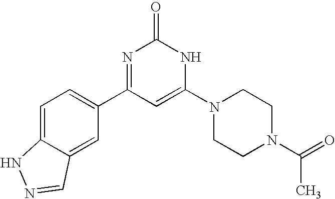 | 6-(4-acetylpiperazin-1-yl)-4- (1H-indazol-5-yl)pyrimidin- 2(1H)-one | 338.4 |
| 26 | 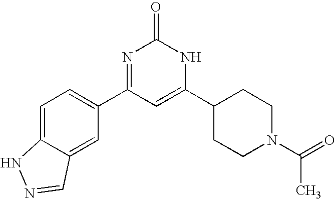 | 6-(1-acetylpiperidin-4-yl)-4- (1H-indazol-5-yl)pyrimidin- 2(1H)-one | 337.4 |
| 27 | 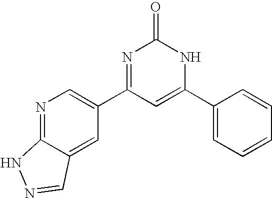 | 6-phenyl-4-(1H-pyrazolo[3,4- b]pyridin-5-yl)pyrimidin-2(1H)- one | 289.3 |
| 28 | 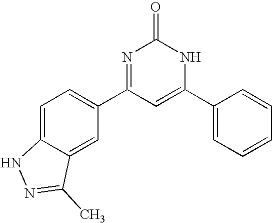 | 4-(3-methyl-1H-indazol-5-yl)- 6-phenylpyrimidin-2(1H)-one | 302.3 |
| 29 | 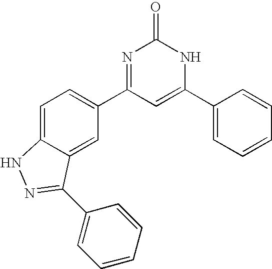 | 6-phenyl-4-(3-phenyl-1H- indazol-5-yl)pyrimidin-2(1H)- one | 364.4 |
| 30 | 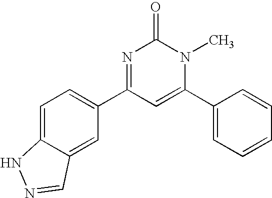 | 4-(1H-indazol-5-yl)-1-methyl- 6-phenylpyrimidin-2(1H)-one | 302.3 |
| 31 | 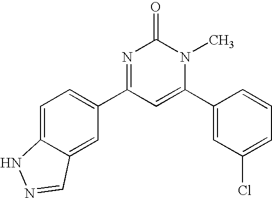 | 6-(3-chlorophenyl)-4-(1H- indazol-5-yl)-1- methylpyrimidin-2(1H)-one | 336.8 |
| 32 | 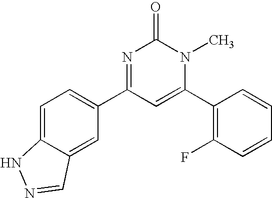 | 6-(2-fluorophenyl)-4-(1H- indazol-5-yl)-1- methylpyrimidin-2(1H)-one | 320.3 |
| 33 | 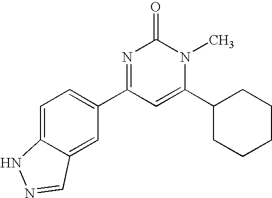 | 6-cyclohexyl-4-(1H-indazol-5- yl)-1-methylpyrimidin-2(1H)- one | 308.4 |
| 34 | 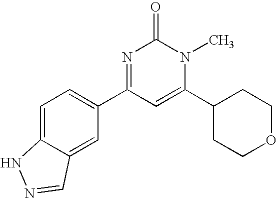 | 4-(1H-indazol-5-yl)-1-methyl- 6-(tetrahydro-2H-pyran-4- yl)pyrimidin-2(1H)-one | 310.3 |
| 35 | 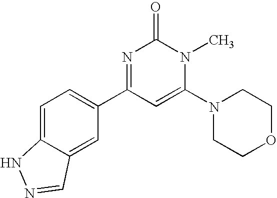 | 4-(1H-indazol-5-yl)-1-methyl- 6-morpholin-4-ylpyrimidin- 2(1H)-one | 311.3 |
| 36 | 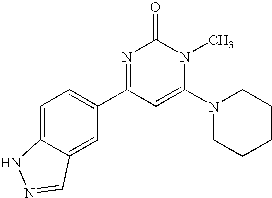 | 4-(1H-indazol-5-yl)-1-methyl- 6-piperidin-1-ylpyrimidin- 2(1H)-one | 309.3 |
| 37 | 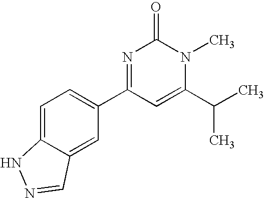 | 4-(1H-indazol-5-yl)-6- isopropyl-1-methylpyrimidin- 2(1H)-one | 268.3 |
| 38 | 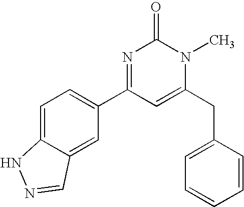 | 6-benzyl-4-(1H-indazol-5-yl)-1- methylpyrimidin-2(1H)-one | 316.3 |
| 39 | 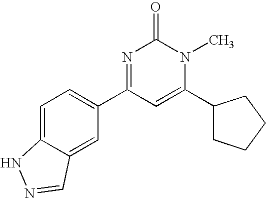 | 6-cyclopentyl-4-(1H-indazol-5- yl)-1-methylpyrimidin-2(1H)- one | 294.3 |
| 40 | 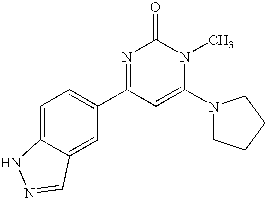 | 4-(1H-indazol-5-yl)-1-methyl- 6-pyrrolidin-1-ylpyrimidin- 2(1H)-one | 295.3 |
| 41 | 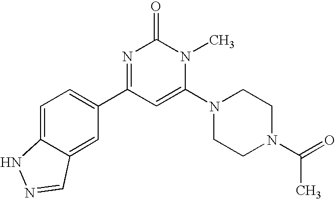 | 6-(4-acetylpiperazin-1-yl)-4- (1H-indazol-5-yl)-1- methylpyrimidin-2(1H)-one | 352.4 |
| 42 | 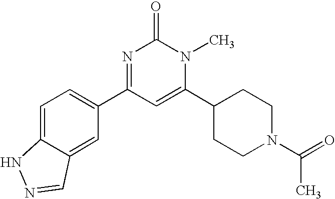 | 6-(1-acetylpiperidin-4-yl)-4- (1H-indazol-5-yl)-1- methylpyrimidin-2(1H)-one | 351.4 |
| 43 | 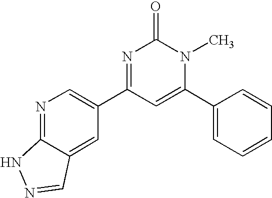 | 1-methyl-6-phenyl-4-(1H- pyrazolo[3,4-b]pyridin-5- yl)pyrimidin-2(1H)-one | 303.3 |
| 44 | 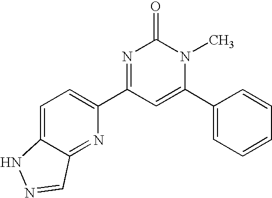 | 1-methyl-6-phenyl-4-(1H- pyrazolo[4,3-b]pyridin-5- yl)pyrimidin-2(1H)-one | 303.3 |
| 45 | 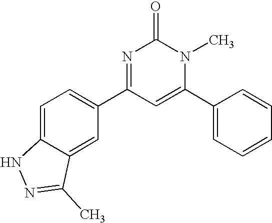 | 1-methyl-4-(3-methyl-1H- indazol-5-yl)-6- phenylpyrimidin-2(1H)-one | 316.3 |
| 46 | 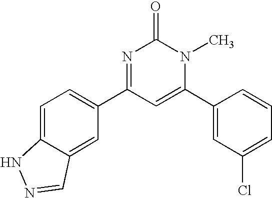 | 6-(3-chlorophenyl)-4-(1H- indazol-5-yl)-1- methylpyrimidin-2(1H)-one | 336.8 |
| 47 | 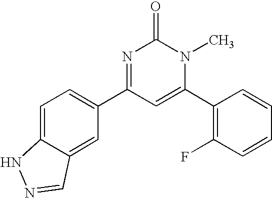 | 6-(2-fluorophenyl)-4-(1H- indazol-5-yl)-1- methylpyrimidin-2(1H)-one | 320.3 |
| 48 | 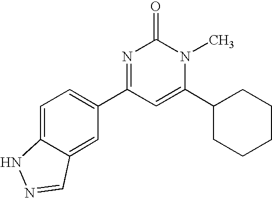 | 6-cyclohexyl-4-(1H-indazol-5- yl)-1-methylpyrimidin-2(1H)- one | 308.4 |
| 49 | 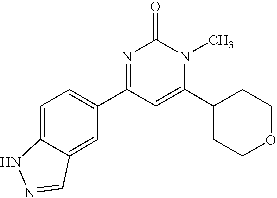 | 4-(1H-indazol-5-yl)-1-methyl- 6-(tetrahydro-2H-pyran-4- yl)pyrimidin-2(1H)-one | 310.3 |
| 50 | 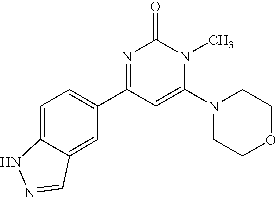 | 4-(1H-indazol-5-yl)-1-methyl- 6-morpholin-4-ylpyrimidin- 2(1H)-one | 311.3 |
| 51 | 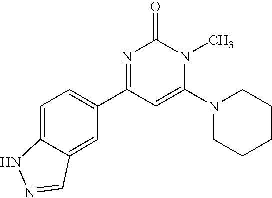 | 4-(1H-indazol-5-yl)-1-methyl- 6-piperidin-1-ylpyrimidin- 2(1H)-one | 309.4 |
| 52 | 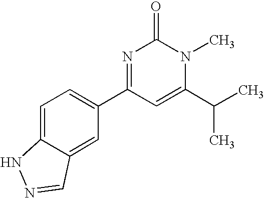 | 4-(1H-indazol-5-yl)-6- isopropyl-1-methylpyrimidin- 2(1H)-one | 268.3 |
| 53 | 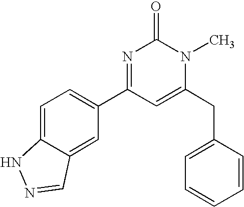 | 6-benzyl-4-(1H-indazol-5-yl)-1- methylpyrimidin-2(1H)-one | 316.3 |
| 54 | 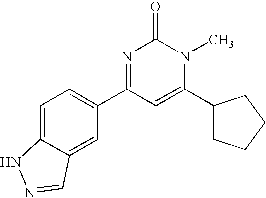 | 6-cyclopentyl-4-(1H-indazol-5- yl)-1-methylpyrimidin-2(1H)- one | 294.3 |
| 55 | 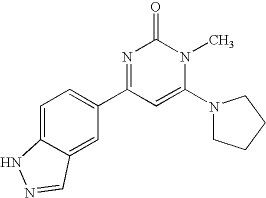 | 4-(1H-indazol-5-yl)-1-methyl- 6-pyrrolidin-1-ylpyrimidin- 2(1H)-one | 295.3 |
| 56 | 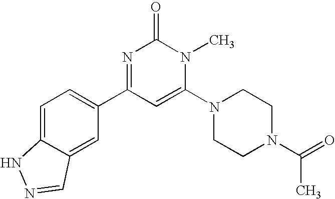 | 6-(4-acetylpiperazin-1-yl)-4- (1H-indazol-5-yl)-1- methylpyrimidin-2(1H)-one | 352.4 |
| 57 | 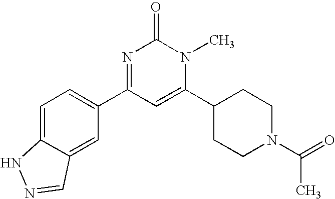 | 6-(1-acetylpiperidin-4-yl)-4- (1H-indazol-5-yl)-1- methylpyrimidin-2(1H)-one | 351.4 |
| 58 | 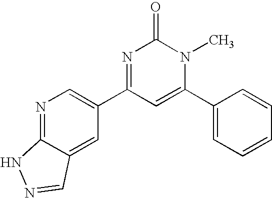 | 1-methyl-6-phenyl-4-(1H- pyrazolo[3,4-b]pyridin-5- yl)pyrimidin-2(1H)-one | 303.3 |
| 59 | 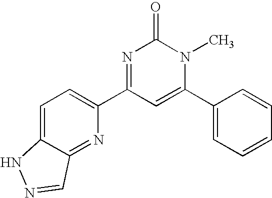 | 1-methyl-6-phenyl-4-(1H- pyrazolo[3,4-b]pyridin-5- yl)pyrimidin-2(1H)-one | 303.3 |
| 60 | 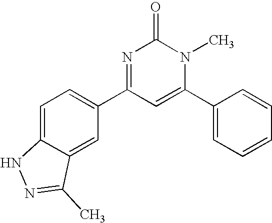 | 1-methyl-4-(3-methyl-1H- indazol-5-yl)-6- phenylpyrimidin-2(1H)-one | 316.4 |
| 61 | 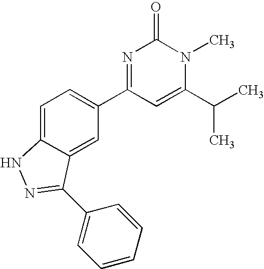 | 6-isopropyl-1-methyl-4-(3- phenyl-1H-indazol-5- yl)pyrimidin-2(1H)-one | 344.4 |
| 62 | 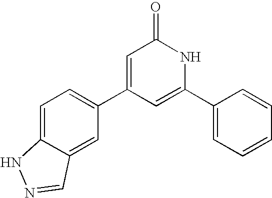 | 4-(1H-indazol-5-yl)-6- phenylpyridin-2(1H)-one | 287.3 |
| 63 | 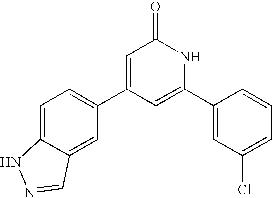 | 6-(3-chlorophenyl)-4-(1H- indazol-5-yl)pyridin-2(1H)-one | 321.8 |
| 64 | 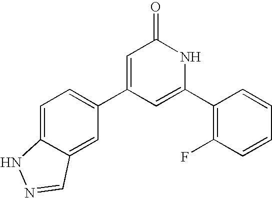 | 6-(2-fluorophenyl)-4-(1H- indazol-5-yl)pyridin-2(1H)-one | 305.3 |
| 65 | 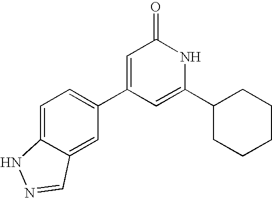 | 6-cyclohexyl-4-(1H-indazol-5- yl)pyridin-2(1H)-one | 293.3 |
| 66 | 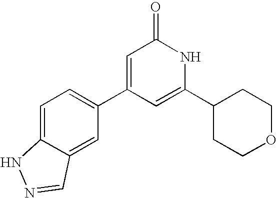 | 4-(1H-indazol-5-yl)-6- (tetrahydro-2H-pyran-4- yl)pyridin-2(1H)-one | 295.3 |
| 67 | 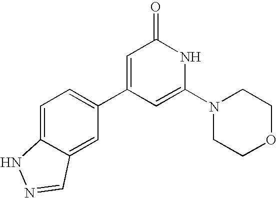 | 4-(1H-indazol-5-yl)-6- morpholin-4-ylpyridin-2(1H)- one | 296.3 |
| 68 | 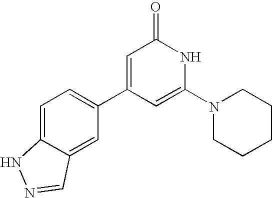 | 4-(1H-indazol-5-yl)-6- piperidin-1-ylpyridin-2(1H)-one | 294.3 |
| 69 | 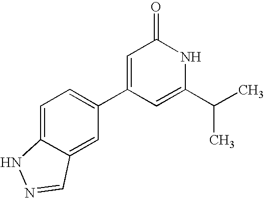 | 4-(1H-indazol-5-yl)-6- isopropylpyridin-2(1H)-one | 253.3 |
| 70 | 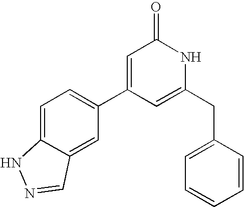 | 6-benzyl-4-(1H-indazol-5- yl)pyridin-2(1H)-one | 301.3 |
| 71 | 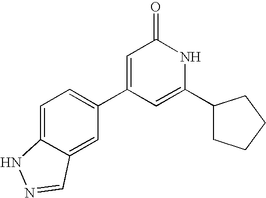 | 6-cyclopentyl-4-(1H-indazol-5- yl)pyridin-2(1H)-one | 279.3 |
| 72 | 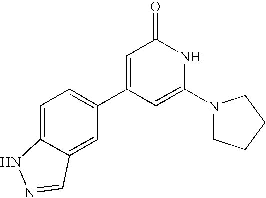 | 4-(1H-indazol-5-yl)-6- pyrrolidin-1-ylpyridin-2(1H)- one | 280.3 |
| 73 | 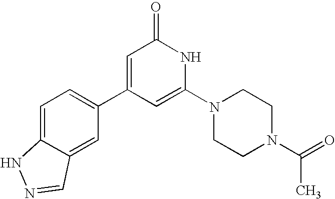 | 6-(4-acetylpiperazin-1-yl)-4- (1H-indazol-5-yl)pyridin- 2(1H)-one | 337.4 |
| 74 | 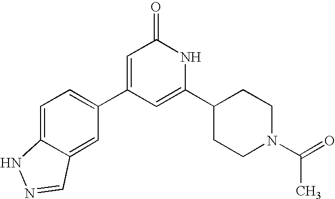 | 6-(1-acetylpiperidin-4-yl)-4- (1H-indazol-5-yl)pyridin- 2(1H)-one | 338.4 |
| 75 | 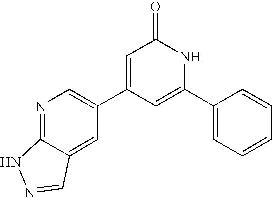 | 6-phenyl-4-(1H-pyrazolo[3,4- b]pyridin-5-yl)pyridin-2(1H)- one | 288.3 |
| 76 | 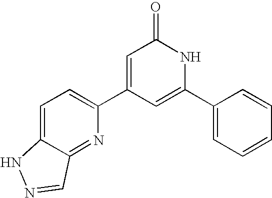 | 6-phenyl-4-(1H-pyrazolo[4,3- b]pyridin-5-yl)pyridin-2(1H)- one | 288.3 |
| 77 | 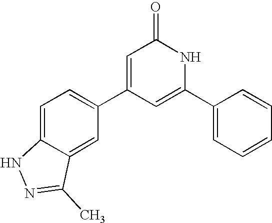 | 4-(3-methyl-1H-indazol-5-yl)- 6-phenylpyridin-2(1H)-one | 302.3 |
| 78 | 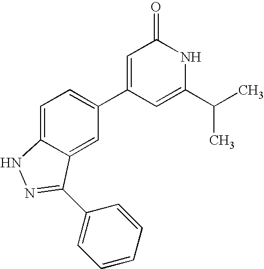 | 6-isopropyl-4-(3-phenyl-1H- indazol-5-yl)pyridin-2(1H)-one | 329.4 |

### Example 79

### In Vitro Assay of CDC7/DBF4 Inhibition

A 20.5 μL kinase reaction was performed on OptiPlate-384 plates PerkinElmer, 6007290) as follows by sequential addition of: 0.5 μL of test compounds of the invention in DMSO, 10 μL 0.5 μM ATP in reaction buffer, 10 μL 2.2 nM cdc7/dbf4 baculovirus derived), 4.4 nM MCM-2 in a reaction buffer. The reaction proceeded for 1 hr at room temperature on an orbital shaker. The reaction was terminated by addition of 10 μL detection buffer containing Streptavidin-coated donor beads and Protein A conjugated acceptor beads (54 μg/ml), and 1:4000 diluted rabbit antibody against phosphoserine 108-MCM-2 (Bethyl Labs). The mixture was incubated at room temperature for 4 hrs in the dark. The plate was then read on a PerkinElmer Fusion instrument. The reaction buffer contained 50 mM Hepes (pH 7.2-7.5), 10 mM MgCl₂, 1 mM dithiothreitol (DTT), leupeptin (10 μg/ml), and bovine serum albumin (BSA) (0.2 mg/ml). The detection buffer contained 25 mM Tris (pH 7.5), 400 mM NaCl, 100mM EDTA, 0.3% BSA, and 0.05% Tween 20.

Representative compounds of the invention which inhibited the kinase reaction >70% in the above cdc7/db4 assay were selected for further analysis and confirmation. Test compounds were diluted in DMSO to a concentration of 0.93 μM or 1.39 μM and 0.5 μL of each test solution was added to wells for assay using the assay conditions and methods as described above. The percentage inhibition of the test compounds of Examples 2-16 was determined to be as shown in Table 3:

TABLE 3

| | | Test Concen- | Inhibition |
|---|---|---|---|
| Example | Compound Name | tration (μM) | (%) |
| 2 | 6-(3-fluorophenyl)-4-(1H-indazol- | 0.93 | 82 |
| | 5-yl)pyrimidin-2(1H)-one | | |
| 3 | 6-(2-fluoro-4-methoxyphenyl)-4- | 0.93 | 81 |
| | (1H-indazol-5-yl)pyrimidin- | | |
| | 2(1H)-one | | |
| 4 | 6-(2,5-dimethoxyphenyl)-4-(1H- | 0.93 | 94 |
| | indazol-5-yl)pyrimidin-2(1H)-one | | |
| 5 | 6-(3-fluoro-4-methoxyphenyl)-4- | 0.93 | 75 |
| | (1H-indazol-5-yl)pyrimidin- | | |
| | 2(1H)-one | | |
| 6 | 6-(4-ethylphenyl)-4-(1H-indazol- | 0.93 | 83 |
| | 5-yl)pyrimidin-2(1H)-one | | |
| 7 | 6-(3,4-dimethoxyphenyl)-4-(1H- | 0.93 | 62 |
| | indazol-5-yl)pyrimidin-2(1H)-one | | |
| 8 | 4-(1H-indazol-5-yl)-6-[3-(tri- | 0.93 | 55 |
| | fluoromethyl)phenyl]pyrimidin- | | |
| | 2(1H)-one | | |
| 9 | 6-(2-fluorophenyl)-4-(1H-indazol- | 0.93 | 87 |
| | 5-yl)pyrimidin-2(1H)-one | | |
| 10 | 6-(3-chlorophenyl)-4-(1H-indazol- | 0.93 | 98 |
| | 5-yl)pyrimidin-2(1H)-one | | |
| 11 | 4-(1H-indazol-5-yl)-6-phenyl- | 1.39 | 98 |
| | pyrimidin-2(1H)-one | | |
| 12 | 6-[3-(benzyloxy)phenyl]-4-(1H- | 1.39 | 98 |
| | indazol-5-yl)pyrimidin-2(1H)-one | | |
| 13 | 4-(1H-indazol-5-yl)-6-(4- | 1.39 | 92 |
| | morpholin-4-ylphenyl)pyrimidin- | | |
| | 2(1H)-one | | |
| 14 | 4-(1H-indazol-5-yl)-6-(4- | 1.39 | 84 |
| | phenoxyphenyl)pyrimidin-2(1H)- | | |
| | one | | |
| 15 | 6-[4-(benzyloxy)phenyl]-4-(1H- | 0.93 | 58 |
| | indazol-5-yl)pyrimidin-2(1H)-one | | |
| 16 | 4-(1H-indazol-5-yl)-6-(4- | 0.93 | 94 |
| | piperazin-1-ylphenyl)pyrimidin- | | |
| | 2(1H)-one | | |

### Example 80

### CDC7 pS108 MCM2 Target Modulation Assay

Cells are plated into 96 well tissue culture plates in 100 uls of cell growth media and incubated overnight at 37° C., 5% CO2. The next day compounds at varying concentrations are added to give a final DMSO concentration of 0.5%. The cells are incubated with compound for 4 hours at 37° C., 5% CO₂. Then the cells are washed with PBS buffer, lysed in 100 μL cell lysis buffer and 25 μLs of cell lysate are added to separate high binding, one spot, MSD 96-well plates (Meso Scale Discovery, MSD, Gaithersburg, Md., USA) and incubated at 4° C. for 1 hour. One plate is used to detect total MCM₂ using the Bethyl rabbit anti-MCM2 (BL248) antibody and the other plate is used to detect phosphorylated MCM2 using the Bethyl rabbit anti-pSer108 MCM2 (BL1539) antibody.

The wells are washed and incubated with primary antibody overnight. After a wash step the secondary antibody (MSD Sulfo-Tag IgG antibody labeled with Ruthenium) is added and incubated for 1 hour at 4° C. The plates are washed 4 times with 1× MSD Tris wash buffer and MSD Read buffer is added to each well (MSD Read Buffer T (4×) with surfactant, dilute to 1.5× with water). The plates are read on the MSD (Meso Scale Discovery) ElectroChemiLuminescent (ECL) plate reader. The read-outs allow the determination of levels of phosphorylation on Ser108 of MCM2 in the presence or absence of agents affecting CDC7 kinase activity in cells.

While illustrative embodiments have been illustrated and described, it will be appreciated that various changes can be made therein without departing from the spirit and scope of the invention.

The embodiments of the invention in which an exclusive property or privilege is claimed are defined as follows:

1. A compound of Formula (I): 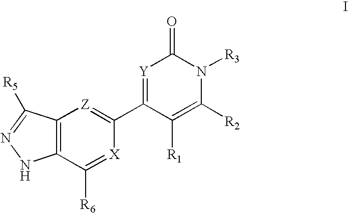
wherein X is N or CR₇;
Y is N or CR₈;
Z is N or CR₄;
R₁ is selected from the group consisting of H, halo, alkyl, substituted alkyl, hydroxy, alkoxy, substituted alkoxy, amino, and substituted amino;
R₂ is selected from the group consisting of alkyl, substituted alkyl, alkoxy, substituted alkoxy, amino, substituted amino, aryloxy, substituted aryloxy, heteroaryloxy, substituted heteroaryloxy, cycloalkyloxy, substituted cycloalkyloxy, heterocyclyloxy, substituted heterocyclyloxy, aryl, substituted aryl, heteroaryl, substituted heteroaryl, cycloalkyl, substituted cycloalkyl, heterocyclyl, and substituted heterocyclyl;
R₃ is H, alkyl, substituted alkyl, aryl or substituted aryl;
R₄, R₆, R₇ and R₈ are independently selected from the group consisting of H, halo, alkyl, substituted alkyl, hydroxy, alkoxy, substituted alkoxy, amino, and substituted amino;
R₅ is selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl; or
a stereoisomer, tautomer, or pharmaceutically acceptable salt thereof.

2. A compound of claim 1, wherein X is CR₇ and Z is CR₄.

3. A compound of claim 1, wherein R₁ is H.

4. A compound of claim 1, wherein R₂ is aryl or substituted aryl.

5. A compound of claim 1, wherein R₃ is H.

6. A compound of claim 2, wherein R₄, R₆, and R₇ are H or halo.

7. A compound of claim 2, wherein R₄, R₆, and R₇ are H.

8. A compound of claim 1, wherein R₅ is selected from the group consisting of H, halo, hydroxy, alkyl, substituted alkyl, amino, substituted amino, alkoxy, and substituted alkoxy.

9. A compound of claim 1, wherein R₅ is H.

10. A compound of claim 1, wherein R₂ is phenyl or substituted phenyl.

11. A compound of claim 1, wherein Y is N.

12. A compound of claim 1, wherein Y is CR₈ and only one of X and Z is N.

13. A compound of Formula (II): 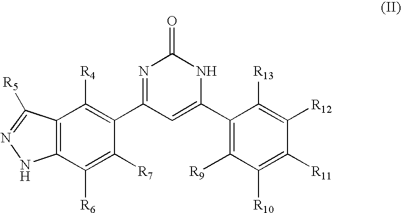
wherein R₄, R₆, and R₇ are independently selected from the group consisting of H, halo, alkyl, substituted alkyl, hydroxy, alkoxy, substituted alkoxy, amino, and substituted amino;
R₅ is selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, and substituted heterocyclyl;
R₉, R₁₀, R₁₁, R₁₂, and R₁₃ are independently selected from the group consisting of H, alkyl, substituted alkyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, sulfonyl, substituted sulfonyl, sulfonyloxy, thioacyl, thiol, alkylthio, substituted alkylthio, aryl, substituted aryl, heteroaryl, substituted heteroaryl, cycloalkyl, substituted cycloalkyl, heterocyclyl, substituted heterocyclyl, aryloxy, substituted aryloxy, heteroaryloxy, substituted heteroaryloxy, cycloalkyloxy, substituted cycloalkyloxy, heterocyclyloxy, and substituted heterocyclyloxy; or
a stereoisomer, tautomer, or pharmaceutically acceptable salt thereof.

14. A compound of claim 13, wherein at least one of R₉, R₁₀, R₁₁, R₁₂, and R₁₃ is alkoxy.

15. A compound of claim 13, wherein at least one of R₉, R₁₀, R₁₁, R₁₂, and R₁₃ is halo, alkyl, or substituted alkyl.

16. A compound of claim 13, wherein R₁₀ is selected from the group consisting of halo, alkyl, substituted alkyl, alkoxy, substituted alkoxy, aryl, substituted aryl, heteroaryl, substituted heteroaryl, cycloalkyl, substituted cycloalkyl, heterocyclyl, substituted heterocyclyl, aryloxy, substituted aryloxy, heteroaryloxy, substituted heteroaryloxy, cycloalkyloxy, substituted cycloalkyloxy, heterocyclyloxy, and substituted heterocyclyloxy.

17. A compound of claim 13, wherein R₄, R₆, and R₇ are H or halo.

18. A compound of claim 13, wherein R₄, R₆, and R₇ are H.

19. A compound of claim 13, wherein R₅ is selected from the group consisting of H, halo, hydroxy, alkyl, substituted alkyl, amino, substituted amino, alkoxy, and substituted alkoxy.

20. A compound of claim 13, wherein R₅ is H.

21. A compound of claim 1 selected from the group consisting of 6-(3-fluorophenyl)-4-(1H-indazol-5-yl)pyrimidin-2(1H)-one, 6-(2-fluoro-4-methoxyphenyl)-4-(1H-indazol-5-yl)pyrimidin-2(1H)-one, 6-(2,5-dimethoxyphenyl)-4-(1H-indazol-5-yl)-pyrimidin-2(1H)-one, 6-(3-fluoro-4-methoxyphenyl)-4-(1H-indazol-5-yl)pyrimidin-2(1H)-one, 6-(4-ethylphenyl)-4-(1H-indazol-5-yl)pyrimidin-2(1H)-one, 6-(3,4-dimethoxyphenyl)-4-(1H-indazol-5-yl)pyrimidin-2(1H)-one, 4-(1H-indazol-5-yl)-6-[3-(trifluoromethyl)phenyl]pyrimidin-2(1H)-one, 6-(2-fluorophenyl)-4-(1H-indazol-5-yl)-pyrimidin-2(1H)-one, 6-(3-chlorophenyl)-4-(1H-indazol-5-yl)pyrimidin-2(1H)-one, 4-(1H-indazol-5-yl)-6-phenylpyrimidin-2(1H)-one, 6-[3-(benzyloxy)phenyl]-4-(1H-indazol-5-yl)pyrimidin-2(1H)-one, 4-(1H-indazol-5-yl)-6-(4-morpholin-4-ylphenyl)-pyrimidin-2(1H)-one, 4-(1H-indazol-5-yl)-6-(4-phenoxyphenyl)pyrimidin-2(1H)-one, 6-[4-(benzyloxy)phenyl]-4-(1H-indazol-5-yl)pyrimidin-2(1H)-one, and 4-(1H-indazol-5-yl)-6-(4-piperazin-1-ylphenyl)pyrimidin-2(1H)-one, or a stereoisomer, tautomer, or pharmaceutically acceptable salt thereof.

22. A compound of claim 1, wherein only one of X and Z is N.

23. A pharmaceutical composition comprising a compound of any one of claims 1-22 and a pharmaceutically acceptable excipient.

24. A method for treating a condition by inhibition of CDC7 activity comprising administering to a patient in need of such treatment an effective amount of a compound of any one of claims 1-22.

25. The method of claim 24 wherein the condition is cancer.

26. The method of claim 25 wherein the cancer comprises cells that express CDC7.

27. A method of inhibiting phosphorylation of MCM2, comprising exposing MCM2, CDC7 and ATP to a compound of any one of claims 1-22.
